(19) United States
(12) Patent Application Publication
(10) Pub. No.: US 2024/0000511 A1
(43) Pub. Date: Jan. 4, 2024

(54) VISUALLY POSITIONED SURGERY

(71) Applicants: Peter Andrew Flaherty, Los Angeles, CA (US); Carlos Andres Uquillas Armas, Redondo Beach, CA (US)

(72) Inventors: Peter Andrew Flaherty, Los Angeles, CA (US); Carlos Andres Uquillas Armas, Redondo Beach, CA (US)

(21) Appl. No.: 18/217,410

(22) Filed: Jun. 30, 2023

Related U.S. Application Data
(60) Provisional application No. 63/357,732, filed on Jul. 1, 2022.

(51) Int. Cl. A61B 34/10; G16H 40/67; A61B 1/00; A61B 1/313; A61B 1/04; A61B 1/06; A61B 5/06; A61L 29/00; A61L 29/14; G06T 7/73; G06V 10/774; G06V 20/50; G06V 10/77; G06V 20/70

(52) U.S. Cl. CPC A61B 34/10; G16H 40/67; A61B 1/000096; A61B 1/00045; A61B 1/313; A61B 1/04; A61B 1/0661; A61B 5/062; A61B 1/0004; A61L 29/005; A61L 29/14; G06T 7/74; G06V 10/774; G06V 20/50; G06V 10/7715; G06V 20/70; A61B 2034/2055

(57) ABSTRACT

Methods and systems for tracking surgical tools in a three-dimensional (3D) space are described. An example method includes receiving image data captured by a single camera of a first surgical instrument, wherein the image data comprises a two-dimensional (2D) image of a second surgical instrument. Using a machine learning model, a three-dimensional (3D) position of the second surgical instrument is determined. The example method further includes determining a feature of the image data based on the 3D position of the second surgical instrument, and outputting an indication of the feature using a user interface.

### CROSS-REFERENCE TO RELATED APPLICATION

This application claims the priority of U.S. Provisional App. No. 63/357,732, which was filed on Jul. 1, 2022, and is incorporated by reference herein in its entirety.

### BACKGROUND

Traditionally, surgical procedures were “open procedures” in which surgeons would make large incisions in the skin of patients, so that the surgeons could directly visualize the physiological structures involved with the procedure. Open procedures, however, carry several risks for patients. Due to these risks, minimally invasive surgical procedures are growing in popularity. During a minimally invasive surgical procedure, a surgeon inserts surgical instruments through a small incision in the skin of the patient. In many cases, minimally invasive surgical procedures are associated with better post-surgical outcomes than open procedures.

Minimally invasive surgical procedures, however, can be challenging. In various cases, a surgical procedure depends on the three-dimensional (3D) physiology of the patient. In open procedures, it may be relatively easy for a surgeon to perceive the 3D physiology of the patient, because the surgeon can directly visualize the operative field. However, in minimally invasive surgical procedures, surgeons often rely on cameras to visualize the operative field. These cameras typically obtain two-dimensional (2D) images of the operative field, which the surgeons can subsequently view on a monitor or other display device. It can be difficult for surgeons to perceive the 3D physiology of the patient based solely off of the 2D images obtained during minimally invasive surgical procedures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The detailed description is described with reference to the accompanying figures. In the figures, the left-most digit(s) of a reference number identifies the figure in which the reference number first appears. The use of the same reference numbers in different figures indicates similar or identical components or features.

FIG. 1 illustrates an example environment for providing positional assistance during a surgical procedure.

FIGS. 2A to 2B illustrate examples of measuring distances within a 3D space based on 2D images of the space captured by a scope.

FIG. 3 illustrates an example of measuring the length of a curve within a 3D space based on 2D images of the space captured by the scope.

FIG. 4 illustrates an example of measuring an angle defined within a 3D space based on 2D images of the space captured by the scope.

FIG. 5 illustrates an example of defining a structure within a 3D space based on 2D images of the space captured by the scope.

FIG. 6 illustrates an example environment for training a predictive model.

FIG. 7 illustrates an example environment for tracking surgical tools in 3D using one or more 2D images.

FIG. 8 illustrates an example process for determining a feature based on 2D images of an operative field.

FIG. 9 illustrates an example process for training an ML model to track the location of a tool based on 2D images of the tool.

FIG. 10 illustrates an example of one or more devices that can be used to implement any of the functionality described herein.

FIG. 11 illustrates the scope apparatus utilized in an Experimental Example.

FIG. 12 illustrates the probe apparatus utilized in an Experimental Example.

FIG. 13 illustrates a training apparatus utilized in this Experimental Example.

FIG. 14A illustrates an output of a user interface showing a depth predicted by a trained ML model and the actual depth measured by a ruler in vivo.

FIG. 14B illustrates an example frame with an overlay indicating a distance between two points defining a chondral defect.

FIG. 15 illustrates a frame depicting a path of a femoral drill guide being drilled from an anteromedial portal.

FIG. 16 illustrates an example of a frame showing the curvature of the lateral aspect of a trochlea of an example cadaveric knee.

FIG. 17 illustrates an example frame showing differences between different tissue types of an example cadaveric knee.

FIG. 18 illustrates example visual differences between healthy and unhealthy tissue.

### DETAILED DESCRIPTION

This disclosure describes various techniques for tracking the location of a surgical tool in 3D space during a surgical procedure. Examples described herein can be used to track the surgical tool in environments that are obscured from direct view by the surgeon, such as during orthoscopic procedures. In some cases described herein, the location of the tool can be accurately identified (e.g., within 1 millimeter (mm)) based exclusively on 2D images of the tool. Accordingly, sophisticated analysis and assistance can be provided to surgeons based on the 3D location of the tool, even in cases wherein the surgeon only has access to a scope equipped with a single 2D camera.

Various types of features can be identified based on the 3D position of the tool. In some implementations, systems and devices described herein can assist the surgeon with tracking an objective distance between two points or along a curved surface. In some cases, the radius of curvature of a physiological structure can be identified. Angles between physiological structures and/or the tool can be derived based on the 3D location of the tool. In some cases, physiological structures may be highlighted, selected, and identified using the tool. These and other features described herein can provide valuable context to the physiology of the operative field, which can greatly simplify the surgical procedure and enhance patient outcomes.

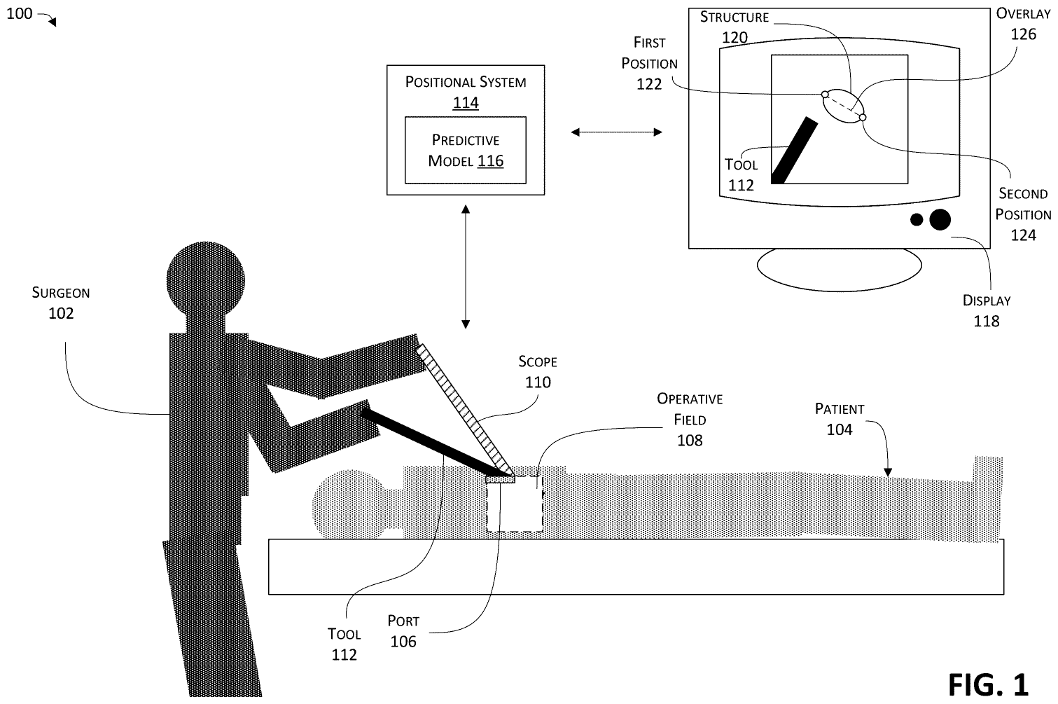

Some particular implementations of the present disclosure will now be described with reference to FIGS. 1 to 18. However, the implementations described with reference to FIGS. 1 to 18 are not exhaustive.

FIG. 1 illustrates an example environment 100 for providing positional assistance during a surgical procedure. The environment 100 includes a surgeon 102 performing a procedure on a patient 104. The surgeon 102 inserts various instruments through a port 106 in the patient 104. In various implementations, the port 106 includes an incision in the skin of the patient 104. Once inserted, the instruments are at least partially disposed in an operative field 108 that is under the skin of the patient 104.

In various cases, the surgeon 102 may limit the size of the port 106. The limited size of the port 106, for instance, may improve surgical outcomes of the patient 104 by minimizing the invasiveness of the procedure. However, the limited size of the port 106 may prevent the surgeon 102 from directly viewing the operative field 108 and/or the instruments disposed in the operative field 108.

To enable the surgeon 102 to visualize the operative field, the instruments may include a scope 110. As used herein, the term “scope,” and its equivalents, may refer to a surgical instrument configured to be at least partially inserted into a body and to capture images of an environment within the body. Examples of scopes include arthroscopes, laparoscopes, endoscopes, and the like. As used herein, the term “image,” and its equivalents, may refer to data representing a collection of pixels or voxels. In various implementations, each pixel of a two-dimensional (2D) image represents a discrete area of a scene being imaged. In various cases, each voxel of a three-dimensional (3D) image (also referred to as a “volumetric image”) represents a discrete volume of a scene being imaged. Each pixel or voxel may be defined by a value that corresponds to a magnitude of a detection signal received from its corresponding area or volume.

As used herein, the term “pixel,” and its equivalents, can refer to a value that corresponds to an area or volume of an image. In a grayscale image, the value can correspond to a grayscale value of an area of the grayscale image. In a color image, the value can correspond to a color value of an area of the color image. In a binary image, the value can correspond to one of two levels (e.g., a 1 or a 0). The area or volume of the pixel may be significantly smaller than the area or volume of the image containing the pixel. In examples of a line defined in an image, a point on the line can be represented by one or more pixels. A “voxel” is an example of a pixel spatially defined in three dimensions.

In various cases, the scope 110 includes a camera configured to capture the images. The camera, for instance, includes an array of photosensors configured to generate two-dimensional (2D) images of the operative field 108. In cases wherein the camera captures multiple 2D images at a predetermined rate, the images may be referred to as “frames” of a video. In some examples, the scope 110 further includes a light source that illuminates the operative field 108 and to enhance the quality of the images captured by the camera. In some cases, the light source directs light through a fiber-optic cable that extends into the operative field 108.

Further, the surgeon 102 may perform the surgical procedure using a tool 112. The tool 112 may be configured to touch, move, cut, puncture, or otherwise manipulate tissues in the operative field 108. Examples of the tool 112 include, for instance, a probe, a cauterizer, a drill, a needle driver, a needle, forceps, suture, a clamp, a suction device, a stapler, a clip, an electrosurgery device, a trocar, a stent, an implant, a saw, a rongeur, or the like.

In various cases, the scope 110 includes a single camera configured to exclusively capture 2D images of the operative field 108. For example, the camera may include a single, 2D array of photosensors configured to generate 2D images of the operative field 108. However, it may be important for the surgeon 102 to perceive the operative field 108 in three dimensions (3D).

In previous environments, the surgeon 102 may be able to perceive the operative field 108 in 3D using one or more techniques. For instance, the surgeon 102 may utilize an alternative camera that captures images of the operative field 108 in 3D. For instance, previous surgical robotic systems may utilize multiple cameras that simultaneously capture 2D images from different angles, and may represent the captured space in 3D using a binocular display. In some alternative cases, the scope 110 could be replaced with an instrument including a depth camera. However, these systems are prohibitively expensive to many practitioners and patients. Accordingly, 3D imaging systems utilizing multiple 2D cameras, depth cameras, or other means for capturing environments in 3D, are not widely utilized for surgical procedures, particularly in low-resource settings.

Another technique for perceiving the operative field 108 in 3D is that the surgeon 102 may manually move the scope 110 around the operative field 108 in order to perceive physiological structures in the operative field 108 from multiple angles. If the surgeon 102 is experienced, this technique may enable the surgeon 102 to perceive the physiological structures in 3D by viewing the 2D images captured by the single camera of the scope 110. However, this technique may be difficult if the surgeon 102 is inexperienced (e.g., if the surgeon 102 is a resident, medical student, or performing a type of surgery that the surgeon 102 has not performed before). Furthermore, in some cases, the physiology of the patient 104 within the operative field 108 may prevent such manipulation of the scope 110. For example, a physiological structure within the operative field 108 may prevent the surgeon 102 from positioning the scope 110 at an angle that will enable the surgeon 102 to appropriately visualize another physiological structure to be manipulated during the surgical procedure. Patient physiology may vary widely, which can make repositioning the scope 110 difficult or impractical depending on the physiology of the patient 104 on which the surgical procedure is being performed.

In various implementations of the present disclosure, a positional system 114 assists the surgeon 102 with perceiving the operative field 108 in 3D using the 2D images captured by the scope 110. The positional system 114 receives the 2D images captured by the scope 110, such as in the form of a stream of 2D images captured by the scope 110 in real-time. The positional system 114 is configured to detect the tool 112 depicted in the 2D images.

In various cases, the 2D images captured by the scope 110 depict the tool 112 in the operative field 108. The relative size of the tool 112 within the 2D images is indicative of the distance between the tool 112 and the camera of the scope 110. A relative size of different parts of the tool 112, as well as the angle of the tool 112 within the frames of the 2D images, are indicative of the orientation of the tool 112 with respect to the scope. Thus, the 3D location and orientation of the tool 112 with respect to the camera of the scope 110 may be derived based on the 2D image, the optics of the camera (e.g., the lenses in the camera used to capture the 2D images), as well as optical ray tracing.

In examples in which the scope 110 is held in a fixed position, the positional system 114 may be able to convert the relative location and orientation of the tool 112 with respect to the scope 110 into the location and orientation of the tool 112 within 3D space using geometry. For instance, the positional system 114 may convert the position and orientation of the tool 112 from a radial coordinate system centered on the camera of the scope 110 to an objective xyz coordinate system that represents the operative field 108. However, in some cases, the position of the scope 110 within the operative field 108 is also variable. As depicted in FIG. 1, the scope 110 is held by the surgeon 102, and can be moved around the operative field 108. Accordingly, it may be challenging to convert the relative 3D location and orientation of the tool 112 into an objective 3D space (e.g., defined by an xyz coordinate system), since the location and orientation of the scope 110 may also be variable.

In various implementations of the present disclosure, the predictive model 116 is configured to process the 2D images from the camera of the scope 110 in order to identify the position and orientation of the tool 112 relative to an objective 3D space. In some cases, the predictive model 116 is configured to identify one or more physiological landmarks within the operative field 108 and depicted by the 2D images. For instance, the physiological landmarks may include at least one of a bone, a blood vessel, an organ, a muscle, a tendon, a ligament, or a tissue in the body of the patient 104. The physiological landmark(s) of the patient 104, for instance, may be assumed to be substantially immobile during the surgical procedure. Based on the identified physiological landmark(s), the predictive model 116 may define a 3D space within the operative field 108. Further, the predictive model 116 may analyze the 2D images in order to determine the position of the tool 112 within the 3D space. In various cases, the predictive model 116 analyzes the 2D images in order to determine the position of the scope 110 within the 3D space. Based on the position of the tool 112 and/or the scope 110, the positional system 114 may provide assistance to the surgeon 102 during the surgical procedure.

The position and orientation of the tool 112 may be determined, by the positional system 114 and the predictive model 116, substantially in real-time. For instance, the positional system 114 may identify the 3D position and orientation of the tool 112 as depicted within a 2D image within 0.1, 0.01, or 0.001 seconds of the positional system 114 receiving the 2D image from the camera of the scope 110. In various cases, the positional system 114 and/or the predictive model 116 may identify the position and orientation of the tool 112 substantially faster than the surgeon 102 could do so without the assistance of the positional system 114 and/or the predictive model 116.

In some examples, the predictive model 116 includes at least one machine learning (ML) model. As used herein, the term “machine learning,” and its equivalents, may refer to a process by which computer-based model that can be used to recognize patterns (e.g., predictive attributes) that the model identifies in training data.

The ML model(s), for instance, include at least one deep learning model. For instance, the predictive model 116 may include at least one convolutional neural network (CNN). A CNN, for instance, is defined according to one or more blocks that are connected to each other in series, in parallel, or a combination thereof. As used herein, the terms “blocks,” “layers,” and the like can refer to devices, systems, and/or software instances (e.g., Application Programming Interfaces (APIs), Virtual Machine (VM) instances, or the like) that generates an output by apply an operation to an input. A “convolutional block,” for example, can refer to a block that applies a convolution operation to an input (e.g., an image). When a first block is in series with a second block, the first block may accept an input, generate an output by applying an operation to the input, and provide the output to the second block, wherein the second block accepts the output of the first block as its own input. When a first block is in parallel with a second block, the first block and the second block may each accept the same input, and may generate respective outputs that can be provided to a third block. In some examples, a block may be composed of multiple blocks that are connected to each other in series and/or in parallel. In various implementations, one block may include multiple layers. In some cases, a block can be composed of multiple neurons. As used herein, the term “neuron,” or the like, can refer to a device, system, and/or software instance (e.g., VM instance) in a block that applies a kernel to a portion of an input to the block. As used herein, the term “kernel,” and its equivalents, can refer to a function, such as applying a filter, performed by a neuron on a portion of an input to a block. The ML model(s), in various cases, is configured to map the tool 112 and/or the scope 110 within 3D space based on the 2D images. In some cases, the ML model(s) is configured to map the physiological structure(s) within the 3D space.

The ML model(s) may be pre-trained with training data. For example, the training data may include 2D images of a training space that omits the operative field 108 and/or ground truth 3D positional data indicating the position and/or orientation of the camera obtaining the 2D images (which, optionally, is the same camera included in the scope 110). As used herein, the terms “position,” “3D position,” and their equivalents, of an object can be represented by a 3D image whose voxels respectively indicate the presence or absence of at least a portion of the object in the volumes represented by the voxels. For example, the 3D position of the camera can be represented based on a 3D image or matrix. As used herein, the term “orientation,” and its equivalents, of an object may refer to an angle of the object with respect to a reference space or plane (e.g., a vertical plane, a horizontal plane, etc.). Further, the 2D images of the training space may depict an instrument (which, optionally, is the tool 112) and the training data may further include ground truth 3D positional data indicating the position and/or orientation of the instrument. In various implementations, the training data includes 2D images depicting the type of physiological structure(s) identified by the positional system 114 in the 2D images from the scope 110 and used to define the objective 3D space in the operative field 108. For instance, the training space may include at least a portion of a tibia of a subject, and the operative field 108 may include at least a portion of a tibia of the patient 104. In some implementations, the ML model(s) are trained in a supervised fashion in order to identify predictive attributes of the 2D images that are indicative of the 3D position and orientation of the camera and the instrument indicated in the training data. These predictive attributes, for instance, include the physiological structure(s) used by the predictive model 116 to define the objective 3D space in the operative field 108.

In various implementations, the predictive model 116 identifies the position of the physiological structure(s) depicted in the 2D images within the objective 3D space. For instance, the predictive model 116 identifies the position of at least one surface of the physiological structure(s), which may be defined in an xyz coordinate system corresponding to the operative field 108. Based on the 2D images and the defined position of the physiological structure(s), the predictive model 116 may further identify the position and orientation of the scope 110 in the objective 3D space, substantially in real-time, even as the scope 110 is being moved around the operative field 108 over time, which impacts the visual field depicted by the 2D images. In addition, the predictive model may identify the position and orientation of the tool 112 in the objective 3D space, substantially in real-time, even as the tool 112 is moved around the operative field 108 over time.

In some cases, the positional system 114 performs additional image processing functions on the 2D images captured by the scope 110, such as before the 2D images are processed by the predictive model 116. For instance, the positional system 114 may adjust a brightness and/or contrast of the 2D images. In some cases, the positional system 114 adjusts a level of at least one channel (e.g., a color channel) of the 2D images. The positional system 114, in some examples, performs edge detection on the 2D images in order to emphasize or otherwise identify boundaries between different types of tissues and structures (e.g., including the structure 120) depicted in the 2D images.

In various implementations, the positional system 114 is configured to output the 2D images captured by the scope 110 on a display 118. For example, the positional system 114 may be configured to visually present a real-time video (e.g., including multiple 2D images captured at a predetermined frame rate) captured by the scope 110. In addition, the surgeon 102 may operate at least one input device in order to cause the positional system 114 to track positional characteristics of the tool 112 and/or other elements within the operative field 108. For instance, the input device may include at least one button disposed on a handle of the scope 110 or the tool 112. In some cases, the input device includes a microphone configured to detect an audible command spoken by the surgeon 102 (e.g., a verbal command, such as “start,” “stop,” etc.). In some implementations, the positional system 114 executes a speech recognition functionality (examples of which are described, for instance, in Nassif et al., IEEEAccess, 9:19143-65 (2019)) to identify the command and associate that identified command with an associated action (e.g., identifying the position of the tool 112 at the time that the command was detected). The positional system 114, in some implementations, outputs an indication of the position and orientation of the tool 112. For instance, in response to the input device detecting an input signal from a user, the positional system 114 may output a pop-up user interface element on the display 118 indicating the position and/or orientation of the tool 112.

Based on the 3D position and orientation of the tool 112, the positional system 114 may be further configured to provide valuable contextual information about the operative field 108 to the surgeon 102 during the surgical procedure. As used herein, such contextual information may be referred to as a “feature” of the 2D image(s), the operative field 108, the scope 110, or the tool 112.

In a particular example, the positional system 114 assists the surgeon 102 with measuring a distance between two points defined by the tool 112 in 3D space. For instance, the positional system 114 determines the length of a structure 120 disposed in the operative field 108. The structure 120, for instance, is depicted in at least one 2D image captured by the scope 110 and visually presented on the display 118. In various cases, the surgeon 102 physically positions the tool 112 at a first position 122 on a first end of the structure 120. The surgeon 102 may indicate the first position 122 by inputting a signal detected by the input device. For instance, the surgeon 102 may press the button and/or call out an audible command that is detected by an input device and indicated to the positional system 114. The surgeon 102 may then move the tool 112 to a second position 124 on a second end of the structure 120. The surgeon 102 may indicate the second position 124 by inputting another signal detected by the input device (e.g., by pressing the button or calling out another audible command).

Once the first position 122 and the second position 124 are defined, the positional system 114 may output information to the surgeon 102 based on the first position 122 and the second position 124. Using the predictive model 116, the positional system 114 may determine the coordinates of the first position 122 and the second position 124 in 3D space based on the position of the tool 112. In some cases, the positional system 114 is configured to determine a distance, within 3D space, between the first position 122 and the second position 124. The positional system 114 may output the determined distance to the surgeon 102. Accordingly, the surgeon 102 may be able to measure the structure 120 within the operative field 108 without the use of a ruler or other, separate measurement device.

The size of structures or other characteristics within the operative field 108 may be clinically relevant in several ways. In a particular example, the surgeon 102 may use these features to measure the width of a physiological structure (e.g., a tumor, a diseased organ, etc.) to be removed from the body of the patient 104. By identifying the size, the surgeon 102 may be able to more effectively select instruments to perform the surgical procedure, or may even be able to determine whether to adjust the size of the port 106 prior to attempting to remove the physiological structure from the body of the patient 104. In some examples, the distance or size of various characteristics in the operative field 108 assist the surgeon 102 with selecting an appropriate treatment. For instance, the positional system 114 may assist the surgeon 102 with measuring a lesion size (e.g., cartilage lesion size) or an amount of bone loss (e.g., in a shoulder).

In various cases, the positional system 114 generates an overlay 126 that indicates the direct segment extending between the first position 122 and the second position 124. In various cases, the surgeon 102 may move the scope 110, thereby shifting the perspective of the operative field 108 depicted on the display 118. The positional system 114 may move the overlay 126 in order to ensure that the overlay 126 depicts the segment between the first position 122 and the second position 124, regardless of the position and orientation of the scope 110. Thus, if the scope 110 is positioned such that it no longer captures 2D images depicting the structure 120, then the overlay 126 may not be presented on the display 118. However, if the scope 110 is repositioned such that it captures 2D images of the structure 120 from a different angle than the angle in which the surgeon 102 selected the first position 122 and the second position 124, then the positional system 114 will make associated adjustments such that the overlay 126 tracks the depictions of first position 122 and the second position 124 on the display 118.

Other types of functions may also be implemented by the positional system 114. For example, the surgeon 102 may define multiple positions along a curved surface in the operative field 108 using the tool 112. The positional system 114 may report the distance along the curved surface. In some cases, the positional system 114 may determine a radius of curvature of the curved surface based on the multiple positions defined along the curved surface. In a particular example, the surgeon 102 may be performing an osteochondral allograft, in which the curvature of the bone is important to consider in order to replace the diseased tissue. The surgeon 102 may therefore mark the surface of the bone using the tool 112, and the positional system 114 may output an indication of the radius of curvature and/or distance along the surface of the bone. In some cases, the surgeon 102 may define multiple positions that indicate an angle within the operative field 108, and the positional system 114 may calculate and output the angle to the surgeon 102.

In some cases, distance measurements can be used to assess the health of a tissue in the operative field 108. For example, “chondromalacia” may refer to damage to hyaline cartilage disposed on a bone surface. Chondromalacia severity is dependent on the mechanical texture of the cartilage. As chondromalacia becomes more severe, for instance, the cartilage becomes more compressible. In some implementations, the structure 120 is a portion of cartilage. The surgeon 102, for instance, may touch the portion of cartilage with the tool 112 and indicate the position to the positional system 114. The surgeon 102 may further compress the cartilage and indicate the compressed position to the positional system 114. In various cases, the positional system 114 may enable the surgeon 102 to assess the state of the cartilage based on the distance between the positions. In some implementations, the tool 112 includes a pressure sensor that can be used to detect a pressure between the tool 112 and the cartilage at the compressed state. For example, the positional system 114 may indicate a compressibility of the cartilage based on the distance and/or the pressure detected by the pressure.

In some cases, the positional system 114 is configured to automatically identify the structure 120 depicted in the 2D images. For example, the surgeon 102 may select the structure 120 by positioning the tool 112 on the structure and providing an input signal to the input device. The positional system 114, in some cases, may perform image segmentation on at least one of the 2D image(s), based on the position of the tool 112 at the that the input signal was detected. For instance, the positional system 114 may perform edge detection in order to identify a boundary of the structure 120 in the operative field 108. In various cases, the positional system 114 may augment the structure 120 as it is depicted on the display 118. For example, the positional system 114 may generate an alternative overlay that highlights the boundary of the selected structure 120.

In some implementations, the positional system 114 is configured to identify the structure 120. For example, the positional system 114 may perform object recognition on the defined boundary of the structure 120. Using various techniques, the positional system 114 may be able to indicate, to the surgeon 102 a health state of the structure 120 (e.g., whether the structure 120 depicts healthy or pathological tissue), a type of the structure 120 (e.g., whether the structure is a blood vessel, an organ, a muscle, a tendon, etc.), or other characteristics about the structure 120. For instance, the predictive model 116 may include a classifier (e.g., an additional machine learning model, such as an additional CNN that has been pretrained to classify physiological structures) configured to identify the structure 120 in the 2D image(s).

According to some implementations, the positional system 114 may calculate and output an angle between a major axis (e.g., a length) of the tool 112 and another line within 3D space. The line may be defined by the surgeon 102, such as using any of the techniques described above. In some cases, the line is defined based on a previously identified structure (e.g., a major axis of the structure 120) in the operative field 108. In a particular example, the surgeon 102 may be performing a multi-ligamentous knee surgery, wherein there are multiple tunnels in a small area of the knee. The angle between these tunnels, for instance, can enable the surgeon 102 to perform drilling without converging, thereby enhancing safety to the patient 104. In another example, the surgeon 102 is preparing to drill a femoral tunnel. The positional system 114, in various cases, may enable the surgeon 102 to drill the femoral tunnel at an appropriate angle that can prevent back wall blow out.

In some cases, the positional system 114 is configured to guide the surgeon 102 with manipulating the tool 112 in the operative field 108. For instance, the positional system 114 may identify a predetermined anatomical landmark (e.g., the structure 120) in the 2D images captured by the scope 110. The positional system 114, in various cases, may further determine the position of the anatomical landmark, such as a position of a surface of the anatomical landmark, in 3D space. Accordingly, the positional system 114 may be configured to determine a relative position of the anatomical landmark with respect to the tool 112. In various implementations, the relative position can be used to assist the surgeon 102 with performing the surgical procedure. For instance, the positional system 114 may output an indication of the relative position (e.g., a pop-up indicating a distance and/or angle between the tool 112 and the anatomical landmark), output directions for the surgeon 102 to safely navigate the anatomical landmark (e.g., may output an instruction to “move right to avoid contact with jugular vein”), or emphasize the anatomical landmark as it is visually presented on the display 118 (e.g., may highlight the anatomical landmark on the display 118). For instance, these features can be used to assist a surgeon 102 with a femoral guide for anterior cruciate ligament reconstruction.

In some cases, the tool 112 is being used during a surgical procedure wherein the positioning of the tool 112 is important. For instance, the tool 112 could be a drill that should be positioned at a specific anatomical position and angle, to avoid damage to certain physiological structures in the operative field 108. In various cases, the positional system 114 is configured to identify a recommended position and/or trajectory of the tool 112 in the 3D space. The positional system 114, for instance, may indicate the recommened position and/or trajectory using one or more user interface elements (e.g., one or more shapes or augmented reality elements) overlaid on the 2D images output by the display 118.

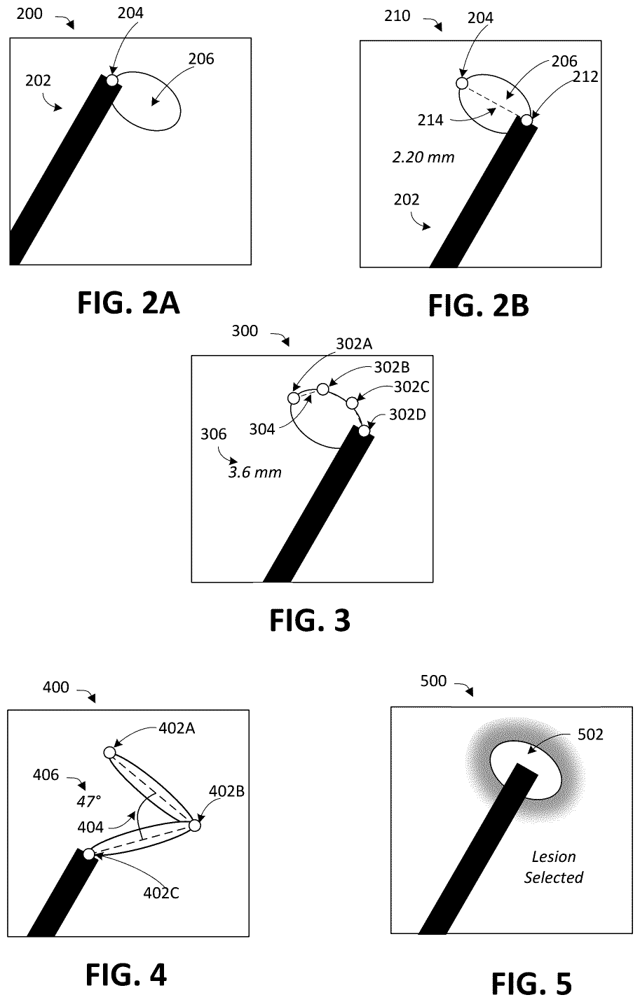

FIGS. 2A to 2B illustrate examples of measuring distances within a 3D space based on 2D images of the space captured by the scope 110. In various implementations, after the surgeon 102 physically positions the tool 112 in the first position 122 in the operative field 108 and provides a command to the positional system 114 to measure a distance between two 3D positions in the operative field 108, the display 118 may display the interface 200 that is depicted in FIG. 2A. For example, before or after positioning the tool 112 in the first position 122, the surgeon 102 may provide a command to the positional system 114 to perform distance measurement. The command may be provided by at least one of pressing a button on tool 112, providing an audible command, or providing a command using a user interface depicted on display 118. The scope 110 may then capture a 2D image of the tool 112 in the operative field 108 and provide the 2D image to the positional system 114. The positional system 114 may then process the received 2D image using the predictive model 116 to determine the first position 122 and use the determined position to generate the interface 200.

As depicted in FIG. 2A, interface 200 includes a user interface element 202 that depicts a 2D rendering of the tool 112 and a user interface element 204 that indicates the first position 122 indicated by the position of the tool 112 within a 2D rendering of the operative field 108 as generated based on image data provided by the tool 112. Interface 200 also includes user interface element 206 that depicts a 2D rendering of the structure 120. The user interface elements 202, 204, and 206 may be generated based on image data captured by the tool 112 at a first time associated with physically positioning the tool 112 at the first position 122.

In various implementations, after providing the distance measurement command and positioning the tool 112 in the first position 122, the surgeon 102 may proceed to physically position the tool 112 in the second position 124 in the operative field 108. The scope 110 may then capture a 2D image of the tool 112 in the operative field 108 and provide the 2D image to the positional system 114. The positional system 114 may then process the received 2D image using the predictive model 116 to determine the second position 124 and use the determined position to generate the interface 210 that is displayed in FIG. 2B. As depicted in FIG. 2B, interface 210 includes, in addition to the user interface elements 202, 204, and 206, a user interface element 212 that highlights the second position 124 indicated by the position of tool 112.

As further depicted in FIG. 2B, interface 210 also includes user interface element 214 that depicts the overlay 126. The overlay 126 may indicate the direct segment extending between the first position 122 and the second position 124. The user interface element 214 may, for example, depict the overlay 126 using an augmented reality (AR) ruler that extends along a line that includes the first position 122 and the second position 124. For example, the ruler's point of origin may be rendered at the first position 122, while a second point of the ruler (e.g., the last point of the ruler) may be rendered at the second position 124 and may indicate the measured distance of the direct segment between the first position 122 and the second position 124. As further depicted in FIG. 2B, interface 210 also includes user interface element 216 that depicts the measured distance associated with the direct segment.

FIG. 3 illustrates an example of measuring the length of a curve within a 3D space based on 2D images of the space captured by the scope 110. In particular, FIG. 3 depicts an interface 300 that assists a surgeon 102 in measuring the length of a curve indicated by placement of the tool 112 within the operative field 108. As depicted in FIG. 3, interface 300 includes user interface 300 that depicts a 2D rendering of the tool 112 and user interface elements 302A-302D that indicate four detected positions of the tool 112 within a 2D rendering of the operative field 108. These user interface elements 302A-302D may be generated based on the image data provided by the scope 110 when the tool 112 is physically positioned at four corresponding points along the curve.

In various implementations, after the surgeon 102 provides the command to measure the length of the curve and positions the tool 112 at the desired positions along the curve, the scope 110 may proceed to capture 2D images of the tool 112 at the desired positions. For example, after placing the tool 112 at each position along the curve, the scope 110 may capture a 2D image of the operative field 108 including the tool 112. Scope 110 may then provide the 2D images to the positional system 114 to determine, using the predictive model 116, 3D positions of the tool 112 in the 2D images. The 3D positions may then be used to generate user interface elements 302A-302D within interface 300.

In various implementations, in addition to computing 3D positions of the tool 112, the predictive model 116 uses the computed 3D positions to determine the length of the curve including the 3D positions. For example, the predictive model 116 may perform one or more computational geometry operations based on the computed 3D positions to determine the corresponding curve length. The interface 300 may then depict a user interface element 304 that is an overlay of a curve segment extending along the computed positions as well as the user interface element 306 that depicts the determined length of the curve segment. The user interface element 304 may visually connect the indicated positions with a continuous curve, providing a visual representation of the path or trajectory along which the tool 112 was positioned.

In various implementations, the user interface element 304 superimposes a measuring tool, such as an AR ruler or an AR tape measure, onto the curve segment. This measuring tool may extend along the curve segment, from the starting position to the ending position. The measuring tool's starting point may be rendered at the first indicated position, while its endpoint (e.g., the last point of the ruler) may be rendered at the final indicated position along the curve segment. The length of the curve segment between these two points may be visually indicated on the measuring tool.

FIG. 4 illustrates an example of measuring an angle defined within a 3D space based on 2D images of the space captured by the scope 110. In various implementations, after the surgeon 102 provides a command to measure an angle indicated by three or more positions in the operative field 108 and positions the tool 112 at the desired positions along the curve, the scope 110 may proceed to capture 2D images of the tool 112 at the desired positions. For example, after placing the tool 112 at each position associated with the angle, the scope 110 may capture a 2D image of the operative field 108 including the tool 112. Scope 110 may then provide the 2D images to the positional system 114 to determine, using the predictive model 116, 3D positions of the tool 112 in the 2D images. The 3D positions may then be used to generate user interface elements 402A-402C in the interface 400 of FIG. 4. As depicted in FIG. 4, each of the user interface elements 402A-402C indicates a 3D position indicated by tool 112 at a point in time.

In various implementations, in addition to computing 3D positions of the tool 112, the predictive model 116 uses the computed 3D positions to determine the angle associated with the 3D positions. For example, the predictive model 116 may perform one or more computational geometry operations based on the computed 3D positions to determine the corresponding angle measurement. The interface 400 may then depict a user interface element 404 that is an overlay of an angle segment characterized by the computed positions as well as the user interface element 406 that depicts the determined measure of the angle. In various implementations, the user interface element 406 superimposes a measuring tool, such as an AR protractor, on the angle.

FIG. 5 illustrates an example of defining a structure within a 3D space based on 2D images of the space captured by the scope 110. In various implementations, after the surgeon 102 provides a command to define a structure and physically positions the tool 112 in a first position of the operative field 108, the scope 110 captures a 2D image of the operative field 108 and provides the captured image to the positional system 114. Afterward, the positional system 114 may use the predictive model 116 to determine the first position based on the received image. The predictive model 116 may also segment the captured image to identify an image segment associated with the first position. The identified segment may then be displayed using the interface 500 depicted by FIG. 5. As shown in FIG. 5, interface 500 presents a user interface 502 that indicates the identified image segment associated with the first position of the tool 112. This segment can represent a particular anatomical structure, such as a blood vessel, a bone, or any other relevant feature within the 3D space.

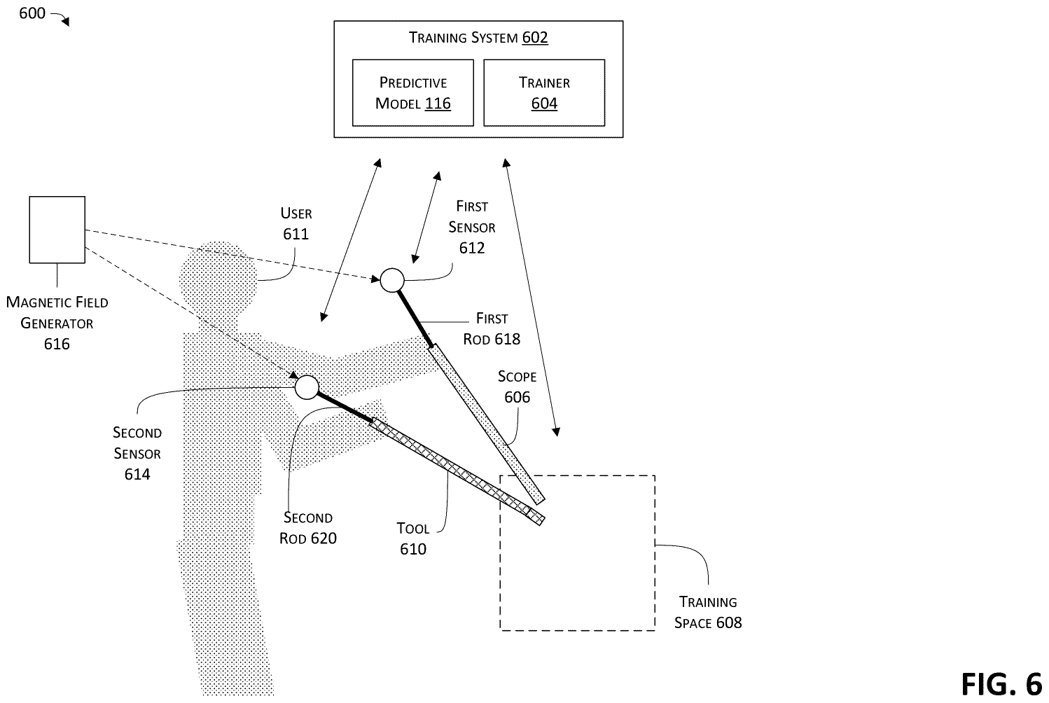

FIG. 6 illustrates an example environment 600 for training the predictive model 116 described above with reference to FIG. 1. In various implementations, a training system 602 is used to train the predictive model 116. The training system 602 includes the predictive model 116 as well as a trainer 604 that optimizes various parameters of the predictive model 116 based on training data.

In various implementations, the training data includes 2D images captured by a scope 606 at least partially disposed within a training space 608. In some cases, the scope 606 is different than the scope 110 described above with reference to FIG. 1. For example, the scope 606 may have a different manufacturer and/or model type than the scope 110. The training space 608, in various cases, is different than the operative field 108 described above with reference to FIG. 1.

In some implementations, the training space 608 includes an interior space of the body of a subject that includes similar physiological features to the operative field 108 of the patient 104. For example, if the operative field 108 is an abdominal space of the patient 104, then the training space 608 may include an abdominal space of a subject who is not the patient 104. In various implementations, the training space 608 includes one or more physiological structures that are similar to one or more physiological structures in the operative field 108. For example, the training space 608 may include at least one of a type of bone, a blood vessel, an organ, a muscle, a tendon, a ligament, or a tissue that is also present in the operative field 108.

Although a single training space 608 is described with reference to FIG. 6, implementations are not so limited. For instance, in some cases, training data may be obtained based on multiple training spaces including the training space 608, such as spaces defined in the bodies of multiple subjects. In some cases, the training data may be obtained based on multiple types of physiological locations. For instance, the training data may include spaces defined in any combination of ears, noses, throats, heads, necks, shoulders, arms, elbows, hands, chests, abdomens, organs, vasculature, legs, knees, feet, or other physiological spaces, in one or more subjects.

A tool 610 is further disposed in the training space 608, such that the 2D images captured by the scope 606 also depict the tool 610. In various cases, the tool 610 is different than the tool 112 described above with reference to FIG. 1. The tool 610, for instance, may be a different type of surgical instrument than the tool 112. In some cases, the tool 610 is a different instance of the same type of surgical instrument as the tool 112. The scope 606 and/or the tool 610 are physically manipulated by a user 611. In some cases, multiple users (including the user 611) hold, move, or otherwise manipulate the scope 606 and the tool 610 in the training space 608 while the training data is obtained.

In various implementations, the training data further includes positional data indicating the ground truth positions and orientations of the scope 606 and tool 610 within the environment 600. The positional data may indicate positions and orientations of the scope 606 and tool 610 within an objective 3D space that represents the training space 608. The positional data, for example, includes xyz coordinates of the scope 606 and the tool 610 simultaneously as the scope 606 is capturing the 2D images. In some cases, the positional data represents the positions and orientations of the scope 606 and tool 610 within a radial coordinate system.

The positional data may include, or at least be derived from, parameters detected by a first sensor 612 and a second sensor 614. In various implementations, the first sensor 612 and the second sensor 614 are magnetic sensors configured to detect a magnetic field emitted by a magnetic field generator 616. For example, the first sensor 612 and the second sensor 614 may communicate (e.g., transmit) data indicative of the detected magnetic field to the training system 602. The training system 602, in various implementations, may be configured to determine the positions and orientations of the first sensor 612 and the second sensor 614 relative to the magnetic field generator 616 based on the detected magnetic field measurements.

Because metallic elements and electronics can generate interference in the magnetic field emitted by the generator 616, as well as the measurements detected by the first sensor 612 and the second sensor 614, the environment 600 may include one or more features to reduce such interference. The scope 606 may include a camera and light source, which can act as a source of interference. Further, the tool 610 may include metallic elements and/or additional electronics that can also act as a source of interference.

In some implementations, the scope 606 and/or the tool 610 include a housing that minimizes the interference. The scope 606 and/or the tool 610 may include at least one electrically insulative material, such as wood, a polymer, an insulative network structure (e.g., an insulative crystal), glass, or any combination thereof. For example, the scope 606 and/or the tool 610 may include a plastic housing that reduces the magnetic interference introduced by the scope 606 and/or the tool 610.

In various implementations, the first sensor 612 and the second sensor 614 are distanced from the scope 606 and tool 610, respectively. This distance can reduce the interference and enhance the accuracy of the positional data. For instance, a first rod 618 is physically coupled to the scope 606 and to the first sensor 612. Similarly, a second rod 620 is physically coupled to the tool 610 and to the second sensor 614. The first rod 618 and the second rod 620 may be made of a rigid and/or insulative material. For example, the first rod 618 and the second rod 620 may include wood, a polymer, an insulative network structure (e.g., an insulative crystal), glass, or any combination thereof. In various cases, the training system 602 may determine the 3D position and orientation of the scope 606 and the tool 610 based on the measurements by the first sensor 612, the measurements by the second sensor 614, the distance and orientation of the first sensor 612 with respect to the scope 606, and the distance and orientation of the second sensor 614 with respect to the tool 610.

In some cases, the 3D positional data of the scope 606 and the tool 610 is preprocessed before inclusion in the training data. For example, the training system 602 may convert the 3D positional data into 2D images.

The training system 602 may be configured to align the 2D images captured by the scope 606 and the 3D positional data. For example, the training system 602 may pair the 2D images with 3D positional data detected simultaneously with the capturing of the 2D images. Thus, the 2D images may be time-aligned with the 3D positional data. According to various cases, the training data includes the 2D images time-aligned with the 3D positional data.

In various cases, the trainer 604 may be configured to train the predictive model 116 based on the training data. The predictive model 116 may be defined according to one or more convolutional layers, each of which is configured to receive an input image, perform a convolution and/or cross-correlation operation on the input image using a kernel (e.g., an image filter), and to provide an output image based on the result of the convolution and/or cross-correlation operation. In various cases, each kernel of each convolutional layer is defined by parameters. For instance, a kernel may be defined according to n by m pixels, wherein n and m are each integer that are greater than 1. The n by m pixels may each have at least one associated value, which is an example of the parameters. The predictive model 116, for instance, may have numerous parameters that are optimized during training.

In various cases, the trainer 604 is configured to optimize the parameters based on the training data. For instance, the trainer 604 may input at least one of the 2D images captured by the scope 606 into the predictive model 116. The predictive model 116 may output data based on the 2D image(s). For example, the convolutional layers of the predictive model 116 may perform their respective convolutional and/or cross-correlation operations on the 2D image(s). Optionally, the predictive model 116 may perform additional operations on the 2D image(s), or data based on the 2D image(s), in order to generate output data. For instance, the predictive model 116, in some cases, performs pooling operations (e.g., max pooling), activation operations (e.g., ReLU activation), and the like.

The output data, in various implementations, is indicative of a predicted 3D position of the scope 606 and/or the tool 610. In various cases, the trainer 604 compares the predicted 3D position of the scope 606 and/or the tool 610 to the ground truth 3D position of the scope 606 and/or the tool 610 at the corresponding time at which the 2D image(s) were obtained. The trainer 604 may calculate a loss (e.g., a difference, variance, or the like) between the predicted 3D position and the ground truth 3D position. Examples of loss include, for example, categorical cross-entropy loss, binary cross-entropy loss, mean squared error (MSE), mean absolute error (MAE), and the like. The trainer 604 may alter the parameters of the predictive model 116 in order to minimize the loss. In various cases, the trainer 604 may train the predictive model 116 by iteratively adjusting the parameters of the predictive model 116 based on the training data.

Once the parameters are optimized based on the training data, the predictive model 116 may be considered trained. In various implementations, the predictive model 116 is represented as data and can be exported to at least one device that does not instantiate the trainer 604.

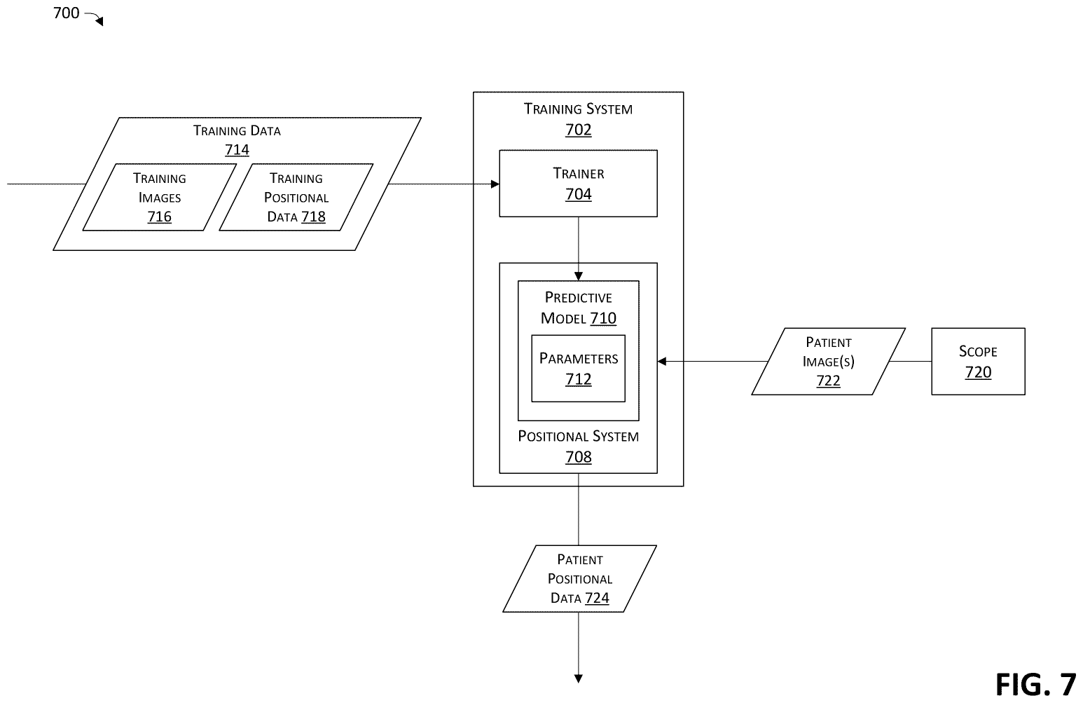

FIG. 7 illustrates an example environment 700 for tracking surgical tools in 3D using one or more 2D images. A training system 702 (e.g., the training system 602) includes a trainer 704 (e.g., the trainer 604) and a positional system 708 (e.g., the positional system 114). The positional system 708 includes a predictive model 710. In various cases, the training system 702 is embodied in hardware, software, or a combination thereof. In various implementations, the predictive model 710 includes one or more deep learning (DL) networks, such as neural networks (NNs) and/or support vector machines (SVMs), that are defined according to parameters 712. The trainer 704 is configured to optimize the parameters 712 of the predictive model 710 based on training data 714.

The training data 714 includes training images 716 and training positional data 718. In various cases, the training images 716 include 2D images of one or more training spaces. The training spaces, for instance, are obtained from interior spaces within bodies of multiple subjects in a population. For instance, the 2D images are obtained from at least one scope that has been inserted into the bodies of the multiple subjects. In some cases, the training spaces include multiple types of spaces, depicting different types of physiological structures, of one or more subjects. In various cases, the training spaces include multiple instances so the same type of physiological space, such as a physiological region (e.g., an abdomen) of multiple subjects. For example, the training spaces may include at least one type of physiological structure, such that the training images 716 depict the type of physiological structure(s). In various cases, the training images 716 depict at least one tool in the training spaces. In various implementations, the training images 716 include frames of one or more videos of the training spaces.

The positional data 718 may indicate the positions and/or orientations of the tool(s) in the training spaces. In some implementations, the positional data 718 indicates the positions and/or orientations of the scope(s) used to capture the training images 716. In some cases, the positional data 718 includes, or is at least derived from, data obtained from a magnetic sensor system, such as the one described above with reference to FIG. 6. In various cases, the positional data 718 indicates the positions and/or orientations of the tool(s) (and optionally the scope(s)) within objective, 3D coordinate systems of each training space.

In some cases, the predictive model 710 includes at least one convolutional neural network (CNN) model. The term “Neural Network (NN),” and its equivalents, may refer to a model with multiple hidden layers, wherein the model receives an input (e.g., an image) and transforms the input by performing operations via the hidden layers. An individual hidden layer may include multiple “neurons,” each of which may be disconnected from other neurons in the layer. An individual neuron within a particular layer may be connected to multiple (e.g., all) of the neurons in the previous layer. An NN may further include at least one fully connected layer that receives a feature map output by the hidden layers and transforms the feature map into the output of the NN.

As used herein, the term “CNN,” and its equivalents, may refer to a type of NN model that performs at least one convolution (or cross correlation) operation on an input image and may generate an output image based on the convolved (or cross-correlated) input image. A CNN may include multiple layers that transforms an input image (e.g., a 3D volume) into an output image via a convolutional or cross-correlative model defined according to one or more parameters. The parameters of a given layer may correspond to one or more filters, which may be digital image filters that can be represented as images. A filter (also referred to as a “kernel”) in a layer may correspond to a neuron in the layer. A layer in the CNN may convolve or cross correlate its corresponding filter(s) with the input image in order to generate the output image. In various examples, a neuron in a layer of the CNN may be connected to a subset of neurons in a previous layer of the CNN, such that the neuron may receive an input from the subset of neurons in the previous layer and may output at least a portion of an output image by performing an operation (e.g., a dot product, convolution, cross-correlation, or the like) on the input from the subset of neurons in the previous layer. The subset of neurons in the previous layer may be defined according to a “receptive field” of the neuron, which may also correspond to the filter size of the neuron. U-Net (see, e.g., Ronneberger, et al., arXiv:1505.04597v1, 2015) is an example of a CNN model. Other examples of CNNs include residual networks (see, e.g., He, et al., arXiv: 1512.03385, 2015 v1), such as ResNet50.

As used herein, the term “CNN,” and its equivalents, may refer to a type of NN model that performs at least one convolution (or cross correlation) operation on an input image and may generate an output image based on the convolved (or cross-correlated) input image. A CNN may include multiple layers that transforms an input image (e.g., a 3D volume) into an output image via a convolutional or cross-correlative model defined according to one or more parameters. The parameters of a given layer may correspond to one or more filters, which may be digital image filters that can be represented as images. A filter in a layer may correspond to a neuron in the layer. A layer in the CNN may convolve or cross correlate its corresponding filter(s) with the input image in order to generate the output image. In various examples, a neuron in a layer of the CNN may be connected to a subset of neurons in a previous layer of the CNN, such that the neuron may receive an input from the subset of neurons in the previous layer and may output at least a portion of an output image by performing an operation (e.g., a dot product, convolution, cross-correlation, or the like) on the input from the subset of neurons in the previous layer. The subset of neurons in the previous layer may be defined according to a “receptive field” of the neuron, which may also correspond to the filter size of the neuron. U-Net (see, e.g., Ronneberger, et al., arXiv:1505.04597v1, 2015) is an example of a CNN model.

In some cases, the predictive model 710 is configured to receive one or more 2D images as input data and generate a 3D image as output data. In some examples, the 2D images are obtained from a single, optical camera. In various cases, the 3D image is defined by an x dimension, a y dimension, and a z dimension, wherein values of the individual voxels in the image indicate the presence or absence of structures, such as physiological structures and surgical instruments (e.g., tools) depicted in the 2D images. In some examples, the values of the individual voxels in the image further indicate the presence or absence of a scope used to generate the 2D images. In various cases, the x dimension, the y dimension, and the z dimension of the 3D image are mapped to an objective 3D space that is being imaged, such that the position and/or orientation of the scope may change (e.g., as additional 2D images are processed and time progresses). Various techniques can be utilized to increase the dimensionality of 2D images in input data, such as computed tomography (CT) and deep learning (see, e.g., Shen et al., Nat. Biomed. Eng. 2019 3(11): 880-88).

In various implementations, the trainer 704 is configured to optimize the parameters 712 of the predictive model 710 based on the training data 714. This process of optimization may be referred to as “training” the predictive model 710. In various cases, the parameters 712 includes values that are modified by the trainer 704 based on the training data 714. For instance, the trainer 704 may perform a training technique utilizing stochastic gradient descent with backpropagation, or any other machine learning training technique known to those of skill in the art. In some implementations, the trainer 704 utilizes adaptive label smoothing to reduce overfitting. According to some cases, the trainer 704 applies L1-L2 regularization and/or learning rate decay to train the predictive model 710.

In various implementations, the trainer 704 may optimize the parameters 712 of the predictive model 710 using a supervised learning technique. For example, the trainer 704 may input the training images 716 into the predictive model 710 and compare outputs of the predictive model 710 to the training positional data 718. The 704 may further modify the parameters 712 (e.g., values of filters in the CNN(s)) in order to ensure that the outputs predictive model 710 are sufficiently similar and/or identical to the training positional data 718.

In particular cases, the 704 is configured to optimize the parameters 712 of the predictive model 710 in order to minimize a loss between an output of the predictive model 710 and the training positional data 718, wherein the predictive model 710 is configured to generate the output based on the training images 716.

Once the predictive model 710 is trained, the positional system 706 may be configured to locate a scope and/or tool in a patient that is omitted from the population used to generate the training data 714. In some cases, data representative of the positional system 706 is exported from the training system 702. For example, a virtual machine (VM) including the positional system 706 may be instantiated on at least one device that is separate from at least one device hosting the trainer 704 and other components of the training system 702.

In various cases, a scope 720 generates one or more patient images 722. The patient image(s) 722, for instance, include at least one 2D image depicting an operative field in an interior space of a body of a patient. In various cases, the operative field of the patient includes one or more types of physiological structures that were depicted in the training images 716. For instance, the interior space of the patient may be the same type of interior space depicted in the training images 716. In some implementations, the patient image(s) 722 further depict a tool in the operative field.

In various implementations, scope 720 includes one or more sensors configured to detect signals (e.g., photons, sound waves, electric fields, magnetic fields, etc.) from the subject being imaged. Further, the scope 720 includes at least one analog to digital converter (ADC) that is configured to convert the signals detected by the sensor(s) into digital data. In various implementations, the scope 720 includes at least one processor configured to generate the patient image(s) 722 based on the digital data.

The trained predictive model 710, for instance, is configured to receive the patient image(s) as an input. In various cases, the patient image(s) exclusively include 2D images detected by a single camera in the scope 720. According to various implementations of the present disclosure, the predictive model 710 is configured to perform one or more operations (e.g., convolution operations, cross-correlation operations, etc.) on the patient image(s) using the parameters 712 optimized during training. The predictive model 710, in various cases, may generate patient positional data 724 based on the patient image(s) 722. That is, the patient positional data 724 may be an output of the predictive model 710 in response to receiving the patient image(s) 722 as an input. In various cases, the patient positional data 724 may indicate the positions and orientations of the scope 720 and the tool depicted in the patient image(s) 722, within a 3D objective space.

In some cases, the positional system 706 outputs the patient positional data 724 to one or more clinical devices. The clinical device(s), for instance, include one or more computing devices associated with at least one care provider, such as a physician associated with the subject. According to some instances, the scope 720 and the clinical device(s) are embodied in a single device. In some cases, the clinical device(s) include a display that visually presents the patient image(s) 722 with at least one user interface element (e.g., a label, a pop-up, a highlight, etc.) representing the patient positional data 724. In some examples, the display shows an overlay representative of a tracked distance, curve, structure, or other element depicted in the patient image(s) 722, on the displayed patient image(s) 722.

FIG. 7 illustrates various components that can be embodied in hardware and/or software. For example, the training system 702, the trainer 704, the positional system 706, the predictive model 710, the scope 720, or any combination thereof, can be implemented in one or more computing devices. That is, one or more of the functions of the training system 702, the trainer 704, the positional system 706, the predictive model 710, the scope 720, or any combination thereof may be executed by at least one processor. The processor(s), in various examples, is configured to execute instructions stored in one or more memory devices, at least one non-transitory computer readable medium, or any combination thereof.

FIG. 7 also illustrates various types of data that is transmitted by or otherwise output by components of the environment 700. Various forms of data described herein can be packaged into one or more data packets. In some examples, the data packet(s) can be transmitted over wired and/or wireless interfaces. For instance, the data may be encoded into one or more communication signals that are transmitted between devices. According to some examples, the data packet(s) can be encoded with one or more keys stored by at least one of the training system 702, the trainer 704, the positional system 706, the predictive model 710, the scope 720, which can protect the data paged into the data packet(s) from being intercepted and interpreted by unauthorized parties. For instance, the data packet(s) can be encoded to comply with Health Insurance Portability and Accountability Act (HIPAA) privacy requirements. In some cases, the data packet(s) can be encoded with error-correcting codes to prevent data loss during transmission.

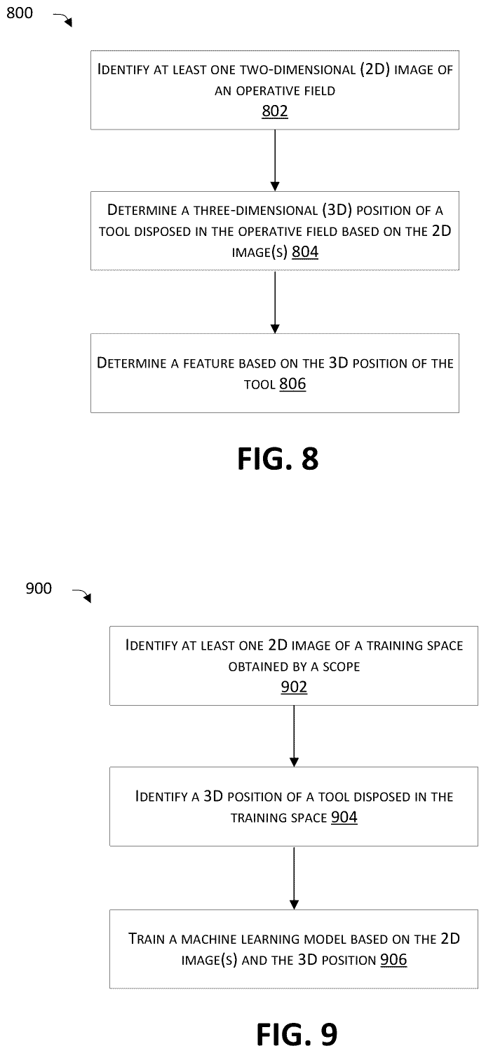

FIG. 8 illustrates an example process 800 for determining a feature based on 2D images of an operative field. The process 800 may be performed by an entity, such as at least one processor, a medical device, an imaging device, the positional system 114, the predictive model 116, the training system 702, the positional system 706, the predictive model 710, or any combination thereof.

At 802, the entity identifies at least one 2D image of an operative field. The 2D image(s) may be captured by a camera (e.g., scope) that is configured to capture images of the operative field. The 2D image(s) may include a 2D image of a tool such as a probe in the operative field.

At 804, the entity determines a 3D position of a tool disposed in the operative field based on the 2D image(s). The 2D image(s) may include a 2D image of a tool such as a probe in the operative field. In various implementations, the entity provides the 2D image(s) to a machine learning model and receives, from the machine learning model, one or more 3D positions of the tool. The machine learning model may have been trained using 2D images of spaces other than the operative field that include a second tool (e.g., a second probe), where the images may be captured by a second camera (e.g., a second scope). The machine learning model may have been trained using sensor data captured by a magnetic field sensor based on positions of a first magnet disposed on a first rod extending from the second tool and positions of a second magnet disposed on a second rod extending from the second camera. The first rod and the second rod may be electrically and magnetically insulative.

At 806, the entity determines a feature based on the 3D position of the tool. The feature may represent at least one of a distance between two 3D positions associated with the tool in the operative field, a measure of an angle between three or more 3D positions associated with the tool in the operative field, a property (e.g., an area) of a region bounded by three or more 3D positions associated with the tool in the operative field, a classification associated with a 3D position associated with the tool in the operative field (e.g., a label of a tissue whose position is defined by the 3D position), a recommended trajectory for moving the tool relative to an anatomical part, a feature of an anatomical label whose position is defined by the 3D position, an orientation of the tool, feedback data about a position of the tool relative to an anatomical part.

In various implementations, determining the feature includes determining a distance between a first 3D position and a second 3D position and determining the feature based on the distance. In various implementations, determining the feature includes determining an angle associated with a first 3D position, a second 3D position, and a third 3D position and determining the feature based on the angle. In various implementations, determining the feature includes determining a region bounded by a first 3D position, a second 3D position, a third 3D position, and a fourth 3D position, and determining the feature based on the region. In various implementations, determining the feature includes determining a classification associated with the 2D image(s) using a second machine learning model and determining the feature based on the classification. The classification may represent at least one of a tissue type associated with a tissue depicted by the 2D image(s), a physiological structure depicted by the 2D image(s), or a pathology depicted by the 2D image(s). In various implementations, the determined feature represents a recommended trajectory for moving the second surgical instrument relative to an anatomical part. In various implementations, the feature represents an orientation of a surgical instrument (e.g., a tool, such as a probe) relative to an anatomical label. In various implementations, the feature represents feedback data about a position of an object (e.g., a tool such as a probe) relative to an anatomical part, where the position of the object may be determined based on the 3D position of a surgical instrument (e.g., a tool such as a probe).

In various implementations, the feature is determined based on a first input signal that is received by an input device when the tool is disposed at a first 3D position and a second input signal that is received by the input device when the tool is disposed at the second 3D position. In various implementations, the input device includes a microphone, the first input signal includes a first verbal command from a user holding the tool and/or the camera, and the second input signal includes a second verbal command from the user.

FIG. 9 illustrates an example process 900 for training an ML model to track the location of a tool based on 2D images of the tool. The process 800 may be performed by an entity, such as at least one processor, a medical device, an imaging device, the positional system 114, the predictive model 116, the training system 702, the positional system 706, the predictive model 710, or any combination thereof.

At 902, the entity identifies at least one 2D image of a training space obtained by a scope. The scope may include at least one of a laparoscope, an orthoscope, or an endoscope.

At 904, the entity identifies a 3D position of a tool disposed in the training space. The entity may identify the 3D position of the tool based on two parameters. In various implementations, the training system includes a first rod extending from the scope and a first sensor configured to detect a first parameter indicative of a 3D position of the first sensor in the training space. The first sensor may be mounted on the first rod and the first sensor may be disposed away from the scope by a first distance. In various implementations, the training system further includes a second rod extending from the tool and a second sensor configured to detect a second parameter indicative of a 3D position of the second sensor in the training space. The second sensor may be mounted on the second rod and the second sensor may be disposed away from the tool by a second distance. The first parameter may include a strength of a magnetic field at the 3D position of the first sensor. The scope may include at least one first metal and the tool may include a second metal. The first rod may include at least one first insulative material and the second rod may include at least one second insulative material. In various implementations, the first insulative material and the second insulative material include at least one of wood or a polymer. At least one of the first or the second distance may be in a range of 15 centimeters (cm) to 30 cm.

In various implementations, the training system includes a magnetic field source configured to emit a magnetic field in the training space. The entity may be configured to determine the 3D position of the first sensor based on a position of the magnetic field source and the strength of the magnetic field at the 3D position of the first sensor. The entity may further be configured to determine the 3D position of the second sensor based on the position of the magnetic field source and the strength of the magnetic field at the 3D position of the second sensor.

At 906, the entity trains the ML model based on the 2D image(s) and the 3D position. The ML model may include at least one of a convolutional neural network, a residual neural network, a recurrent neural network, or a two-stream fusion network comprising a color processing stream and a flow stream. The entity may perform a training technique utilizing stochastic gradient descent with backpropagation, or any other machine learning training technique known to those of skill in the art. In some implementations, the entity utilizes adaptive label smoothing to reduce overfitting. According to some cases, the entity applies L1-L2 regularization and/or learning rate decay to train the predictive model 710.

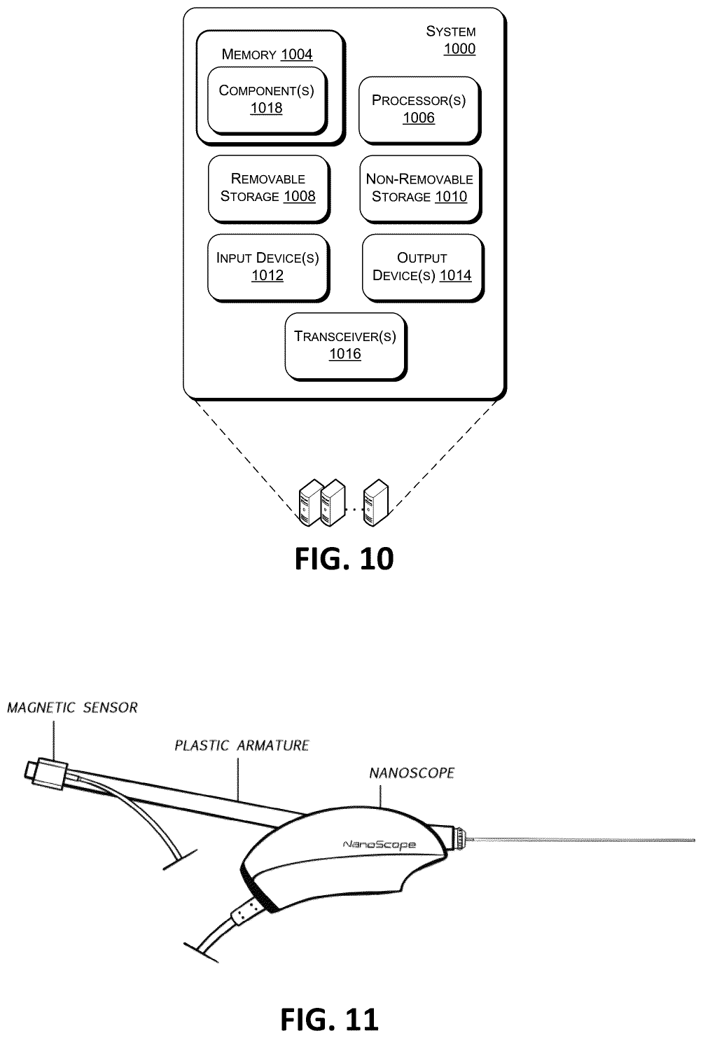

FIG. 10 illustrates an example of one or more devices 1000 that can be used to implement any of the functionality described herein. In some implementations, some or all of the functionality discussed in connection with FIGS. 1-9 can be implemented in the device(s) 1000. Further, the device(s) 1000 can be implemented as one or more server computers 1002, a network element on a dedicated hardware, as a software instance running on a dedicated hardware, or as a virtualized function instantiated on an appropriate platform, such as a cloud infrastructure, and the like. It is to be understood in the context of this disclosure that the device(s) 1000 can be implemented as a single device or as a plurality of devices with components and data distributed among them.

As illustrated, the device(s) 1000 include a memory 1004. In various embodiments, the memory 1004 is volatile (such as RAM), non-volatile (such as ROM, flash memory, etc.) or some combination of the two.

The memory 1004 may store, or otherwise include, various components 1006. In some cases, the components 1006 can include objects, modules, and/or instructions to perform various functions disclosed herein. The components 1006 can include methods, threads, processes, applications, or any other sort of executable instructions. The components 1006 can include files and databases. For instance, the memory 1004 may store instructions for performing operations of any of the scope 110, the positional system 114, the predictive model 116, the training system 602, the trainer 604, or any combination thereof.

In some implementations, at least some of the components 1006 can be executed by processor(s) 1008 to perform operations. In some embodiments, the processor(s) 1008 includes a Central Processing Unit (CPU), a Graphics Processing Unit (GPU), or both CPU and GPU, or other processing unit or component known in the art.

The device(s) 1000 can also include additional data storage devices (removable and/or non-removable) such as, for example, magnetic disks, optical disks, or tape. Such additional storage is illustrated in FIG. 1000 by removable storage 1010 and non-removable storage 1012. Tangible computer-readable media can include volatile and nonvolatile, removable and non-removable media implemented in any method or technology for storage of information, such as computer readable instructions, data structures, program modules, or other data. The memory 1004, removable storage 1010, and non-removable storage 1012 are all examples of computer-readable storage media. Computer-readable storage media include RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information, and which can be accessed by the device(s) 1000. Any such tangible computer-readable media can be part of the device(s) 1000.

The device(s) 1000 also can include input device(s) 1014, such as a button, keypad, a cursor control, a touch-sensitive display, voice input device (e.g., a microphone), etc., and output device(s) 1016 such as a display, speakers, printers, etc. In some implementations, the input device(s) 1014, in some cases, may include a device configured to detect input signals from a user (e.g., a surgeon).

As illustrated in FIG. 10, the device(s) 1000 can also include one or more wired or wireless transceiver(s) 1016. For example, the transceiver(s) 1016 can include a Network Interface Card (NIC), a network adapter, a Local Area Network (LAN) adapter, or a physical, virtual, or logical address to connect to the various base stations or networks contemplated herein, for example, or the various user devices and servers. The transceiver(s) 1016 can include any sort of wireless transceivers capable of engaging in wireless, Radio Frequency (RF) communication. The transceiver(s) 1016 can also include other wireless modems, such as a modem for engaging in Wi-Fi, VViMAX, Bluetooth, or infrared communication.

### Experimental Example 1: Training a ML Model to Identify 3D Position of a Probe in a Knee Based on 2D Frames

This Experimental Example reports the creation of visually positioned surgical software that uses a millimeter-accurate 3D data set captured alongside the video feed from the NANOSCOPE™ at 16.7 ms per frame to train an example ML model to accomplish the same software calculations in the surgical setting in real-time.

During training, ground truth positional data was obtained using a VIPER™ magnetic tracking system (from Polhemus of Colchester, VT), due to its millimeter accuracy, size, and effectiveness. A NANOSCOPE™ (from Arthrex, Inc. of Naples, FL) was selected as a scope. The NANOSCOPE™ has a plastic housing and a limited magnetic footprint. A disposable arthroscopic probe with a relatively low metallic content and a plastic handle was selected as a reference tool.

FIG. 11 illustrates the scope apparatus utilized in this Experimental Example. To further reduce interference that would prevent accurate positional tracking of a magnetic sensor, a plastic armature was attached to the scope. The plastic armature is configured to hold the magnetic sensor at a fixed distance from the scope, but to prevent interference between the magnetic sensor and the electronics of the NANOSCOPE™.

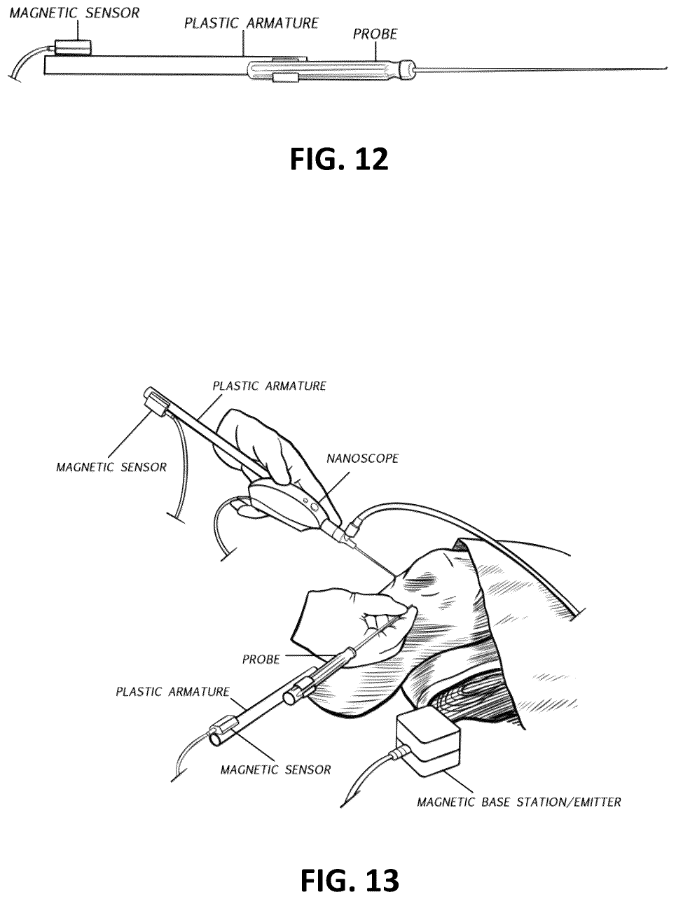

FIG. 12 illustrates the probe apparatus utilized in this Experimental Example. Similarly to the scope apparatus illustrated in FIG. 11, a plastic armature was attached to the probe in order to hold the magnetic sensor at a fixed distance from the probe. The distance between the magnetic sensor and the probe reduced electromagnetic interference between the magnetic sensor and the probe, thereby enhancing the accuracy of the signals detected by the magnetic sensor.

FIG. 13 illustrates a training apparatus utilized in this Experimental Example. As illustrated, a user manually operated the scope apparatus and the probe apparatus. A magnetic base station/emitter was configured to emit a magnetic field in a training space that included a human cadaver knee. The magnetic sensors attached to the scope and probe detected the magnetic field emitted from the magnetic base station/emitter. Based on the magnetic field detected by the emitters, the positions and orientations of the scope and probe apparatuses in 3D space were determined. Further, the scope was configured to capture 2D images of the training space. The 2D images, for instance, depicted at least a portion of the probe apparatus. A training data set was obtained as the user moved the scope apparatus and the probe apparatus throughout the training space. The training data set, for instance, included 2D images captured by the scope apparatus as well as the 3D positions and orientations of the scope apparatus and the probe apparatus.

A calibration procedure was developed for the VIPER™ magnetic tracking system to ensure accurate, consistent readings. The VIPER™ magnetic tracking system was sensitive to position and interference. The magnetic sensors were tested to ensure that their position and orientation were accurately represented and tracked by homing them to the magnetic base station using proprietary software associated with the VIPER™ magnetic tracking system and verifying that they produced an XYZ reading of (0,0,0) in 3D positional space.

A custom 3D software was developed using the UNITY game engine software (from Unity Technologies of San Francisco, CA) in order to allow visualizion of the magnetic sensors in 3D space. The UNITY game engine software is modular and offered a framework for creating custom 3D applications. The VIPER™ Serial Development Kit (SDK) was integrated with the 3D visualizer. This integration was performed by successfully installing the VIPER™ SDK in UNITY. Additional C #code was developed that allowed UNITY to recognize incoming data from the VIPER™ SDK and thereby ingest real-time data produced by the VIPER™ sensor system. In addition, am error-checking methodology was developed to ensure accurate results, successful capture, and integration into the training data set. These error checking methodologies included: (a) placing a duplicate sensor adjacent to the primary sensors (e.g., the sensors included in the scope apparatus and the probe apparatus) and comparing the outputs to confirm that they were receiving the same positional information; and (b) manually confirming the position of the probe in the software at different intervals using a ruler held in the training space. Varied observable test distances outside the anatomy were measured, and used to confirm that the sensors were sub-millimeter accurate.

The position of the probe apparatus was marked in the software at different intervals on a ruler in vivo (i.e., inside the anatomy). Varied observable test distances were measured outside the anatomy in order to verify that the positions detected by the sensors were sub-millimeter accurate.

Video frames were also captured by the scope apparatus and stored with timecodes. The video frames were captured at a frame rate of 60 frames per second (fps), wherein each frame represented 16.7 milliseconds (ms). Using software, the video frames were paired with associated positional data from the sensors with extremely low latency. The data was formatted into an ML-ready dataset. Software (developed using C #) was developed to create sequential filenames for each captured video frame and to insert the filenames into a spreadsheet containing the 3D positional data. Software was also developed to simultaneously ingest magnetic positional data incoming from the sensors into the spreadsheet, thereby correlating each video frame and filename with its respective 3D positional data. The operational order, for instance, prioritized insertion into the spreadsheet before forwarding the data into the UNITY game engine software to assure minimum latency upon ingestion.

A User Interface (UI) was created in the UNITY game engine software that allowed for the operation of the above functions, including buttons for all listed actions. The UI visualized various functional icons representing different parts of the surgical system in a 3D coordinate system. Functional icons for all elements in the surgical system were created, including but not limited to the magnetic base station, sensors, scope, and probe. The UI was also designed to enable addition of other surgical tools that could be added at a later time, for example, drills, debriders, suturing devices, etc. This allowed for a visualization of the magnetic positioning system in 3D software. The visual icons represented objects to be visible in relation to each other in 3D virtual space. Further, the UI enabled data visualization, as well as features enabling measurements and/or recording sessions by users.

The training space utilized in this Experimental Example included an anatomical location. A knee was selected as the anatomical location, because knees are relatively anatomically consistent between subjects. Further, the knee was relatively easy to position for training data acquisition. Notably, the system is suitable for training using other types of anatomical sites, such as the shoulder, wrist, hip, ankle, abdomen, or the like.

To enhance reproducibility, each cadaveric knee was positioned in flexion. In particular, each cadaveric knee was positioned without metallic posts, clamps, or other equipment that could cause significant interference with the readings by the magnetic sensors. Rather, each cadaveric knee was held in a static, flexed position using cloth and plastic equipment. The flexion in each knee provided several benefits including stable and reproducible anatomy, several tissue types visible within a single visual field of the scope, clinically relevant measurements of interest, and enabling the training data set to be obtained without repositioning. The training data set was obtained using multiple cadaveric knees, in order to mimic anatomical variation of a broad population of subjects. The cadaveric knees were obtained from subjects with different biological sex, age, ethnic origin, height, and weight.

Non-reliable soft tissue and fat pad was debrided to optimize visibility of desired anatomical landmarks and structures during training data set acquisition. Before and during training data set acquisition, a suction pump was utilized. Flow and suction parameters were optimized to prevent the creation of floating fat particles and/or bubbles that would distort the training data set. Further, the suction pump was used sparingly to avoid magnetic interference with the sensors.

The scope apparatus and probe apparatus were used to explore each cadaveric knee. Video frames and correlated positional data were obtained for each cadaveric knee. The data was segmented by anatomical compartment. For instance, video frames and positional data corresponding to the intercondylar notch were separated from video frames and positional data corresponding to other anatomical compartments in the knee.

Individual segments of the training data were used to train a ML model. A visual difference calculation on adjacent image pairs was used to determine the change in individual pixels. Accordingly, movement of the structures depicted in the frames (and movement of the scope apparatus itself) was tracked over time. 3D space data of selected portions was transformed into a single linear distance for measurement. This transformation was used to convert the 3D space data into 2D data. The ML model was therefore trained in a supervised manner using 2D data as input (e.g., the frames from the scope) and 2D data as output (i.e., the transformed version of the 3D space data). The ML model utilized in this Experimental Example was ResNet-50.

Once trained, the ML model was able to identify the location and orientation of a probe, within millimeters of the probe's ground truth position, without reliance on the magnetic tracking system. This software offers a critical digital toolkit that a surgeon can utilize in real-time. This offers distinct advantages, most notably not having to rely on external hardware which can be costly, cumbersome to set up, and unreliable in the real-world operating room due to multiple areas of interference.

### Experimental Example 2: Utilizing the Trained ML Model to Track Instruments in 3D Using 2D Frames

Once trained, the ML model described above with reference to Experimental Example 1 was able to identify the location and orientation of a probe, within millimeters of the probe's ground truth position, without reliance on the magnetic tracking system. This software offers a critical digital toolkit that a surgeon can utilize in real-time. This offers distinct advantages, most notably not having to rely on external hardware which can be costly, cumbersome to set up, and unreliable in the real-world operating room due to multiple areas of interference. The ML model was utilized to identify anatomical landmarks; mark, tag, and sort anatomical landmarks; measure the distance between two points; measure the distance between a series of points; measure angles; measure area; measure curvature; identify the orientation of surgical instruments relative to the identified anatomical landmarks; distinguish tissue types; distinguish diseased from healthy tissue; orient and guide the surgical instruments relative to the identified anatomical landmarks; and provide feedback and assessment for the surgeon after the surgical procedure is complete. In this Experimental Example, several tests were performed without the sensors attached to the scope and probe. These tests confirm that the trained ML model can be used to

### Measure Distance Between Two Points

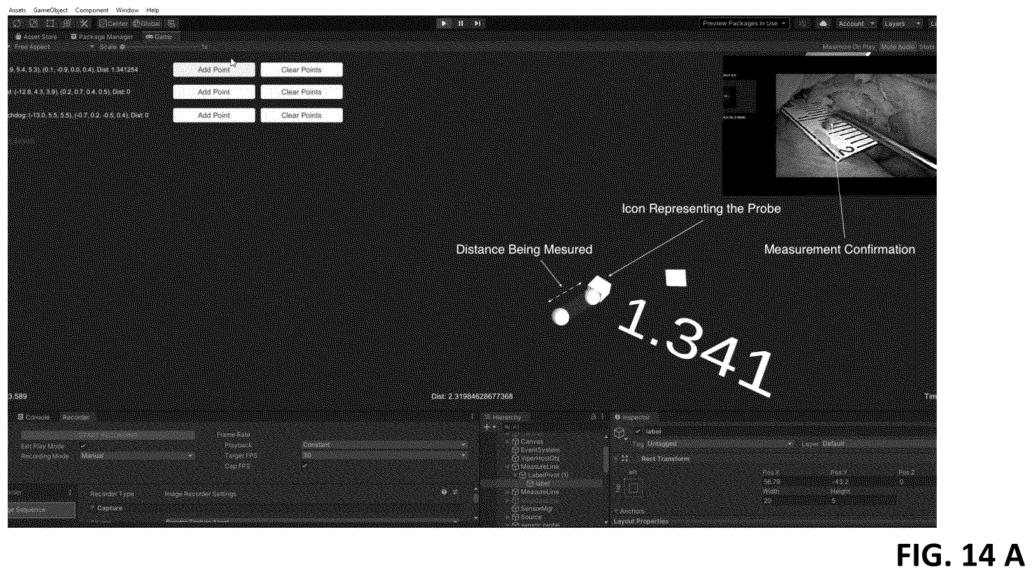

Measuring distance between two points is clinically significant in many ways. In the knee, it can be used to categorize pathology such as cartilage lesion size and this drives treatment. It can help find important anatomic landmarks that have distance relations to other known landmarks. In the shoulder, it can be used to measure bone loss, and this can drive treatment options. In this Experimental Example, the trained ML model was able to accurately identify a length between two points defined along a ray that extended along a depth direction (e.g., substantially parallel to the scope apparatus). The scope and probe were inserted into an example cadaveric knee. Frames captured by the scope were input into the trained ML model. Two positions were indicated by the probe. The trained ML model generated a predicted depth based on the distance between the two positions and the frames captured by the scope. A ruler was used to confirm that the predicted depth was within a mm of an actual depth between the indicated points. FIG. 14A illustrates an output of the UI showing the depth predicted by the trained ML model and the actual depth measured by a ruler in vivo. This test demonstrates measurements by the system in Z-space (depth), which is especially complex, because it is difficult to ascertain from a two-dimensional video image. This indicates highly accurate 3D data.

FIG. 14B illustrates an example frame with an overlay indicating a distance between two points defining a chondral defect. The scope and probe were inserted into an example cadaveric knee. Using the probe, a user defined ends of the chondral defect using the UI described in Experimental Example 1. The system was able to accurately measure the chondral defect using the trained ML model and the frames obtained by the scope.

### Measure Distance Between a Series of Points

Often, cartilage lesions are not linear and do not fit into perfect shape models. By measuring between multiple points, this can allow for measurements of complex shapes. The scope and probe were inserted into an example cadaveric knee and used to define four positions along a curved surface in the knee. The trained ML model was used to predict the distance along the curved surface. The predicted distance was confirmed to be accurate within a millimeter.

### Measure Angle

Angles are clinically significant in many surgeries. One clinical example is during multi-ligamentous knee surgery where there are several different tunnels in one small area. The angle between these tunnels can help facilitate drilling without converging. This allows for safer and more proficient surgery. Another example is the angle at which a femoral tunnel is drilled. Improper angles can lead to back wall blow out, a common and serious surgical complication.

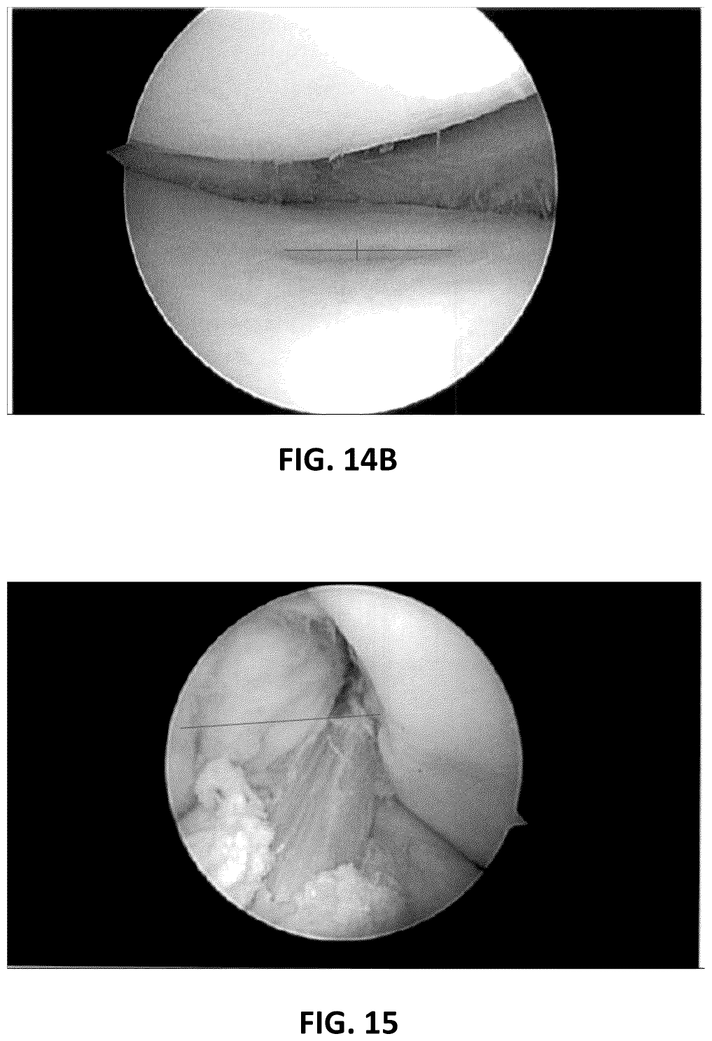

FIG. 15 illustrates a frame depicting a path of a femoral drill guide being drilled from an anteromedial portal. The angle is relevant to preventing back wall blow out, a common complication in ACL surgery. In this test, the ML model was able to identify the path (e.g., as indicated by the line on FIG. 15), and overlay the path on the frame. Further, the trained ML model was able to accurately identify a relative angle between the scope and the probe.

### Measure and Characterize Curvature

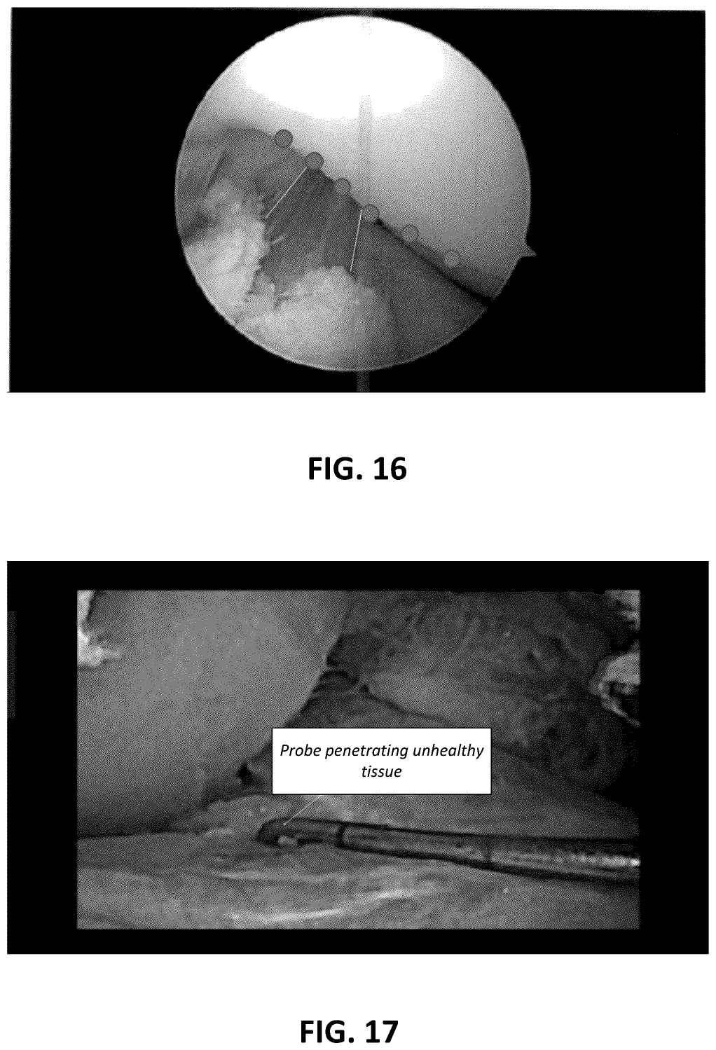

During cartilage restoration procedures, the curvature of the bone is relevant. While replacing the diseased tissue, matching the radius of curvature can improve patient outcomes. In an osteochondral allograft, there are several areas that have different curvatures and being able to accurately match these would allow for improved outcomes. FIG. 16 illustrates an example of a frame showing the curvature of the lateral aspect of a trochlea of an example cadaveric knee. As shown, several user interface elements (visualized as dots) illustrate the curvature.

### Identify the Orientation of Instrument Relative to Anatomical Landmarks

Surgical instrumentation has defined parameters that allow for calculation of orientation once the instrument has been recognized. An example of this would be with a femoral guide for anterior cruciate ligament reconstruction. Using an over-the-top guide, the starting point can be recognized, however the path is much more subtle leading to the potential for back wall blow out. By recognizing the orientation and location of the instrument in relation to the relevant anatomy (back wall), a safe trajectory can be obtained with reassurance.

### Distinguishing Tissue Types

FIG. 17 illustrates an example frame showing differences between different tissue types of an example cadaveric knee. For example, the knee includes bone, cartilage, meniscus, and ligament. These tissues have different positional, visual, and mechanical characteristics that can be distinguished using the trained ML model.

Distinguishing Diseased from Healthy Tissue

In a test, the probe was used to penetrate into unhealthy cartilage tissue. Cartilage health can be categorized by the depth of wear. Using the depth that a probe can penetrate cartilage can help distinguish which category of chondromalacia it is.

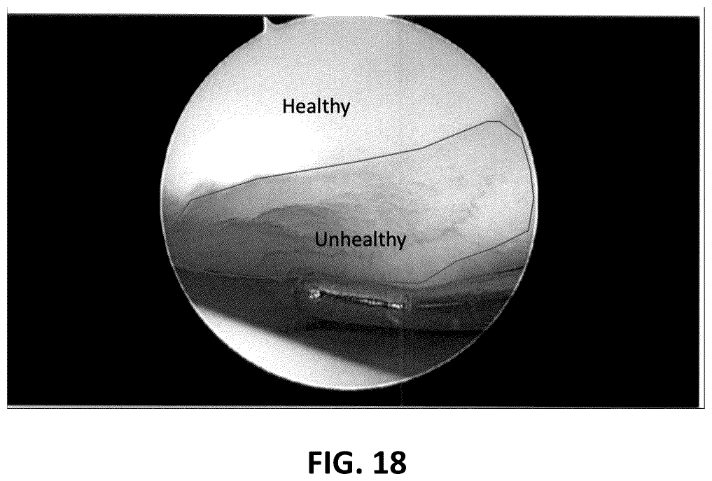

FIG. 18 illustrates example visual differences between healthy and unhealthy tissue. Tissue health can be distinguished visually by measuring the distance a probe is able to go into the cartilage. Healthy tissue can also be ascertained using visual markers on the tissue itself. By applying visual filters within the computer vision system such as Brightness/Contrast, Levels, and Find Edges, automated notifications to the surgeon using the trained ML model can be performed on a purely visual basis also.

### Providing Feedback and Assessment After Procedure is Complete

Feedback and task checking are an important part of reproducibility in surgery. For example, once a guide pin has been inserted for a tunnel during ACL surgery, the position of the pin can be evaluated to provide feedback whether the position is optimal for improved outcomes.

### EXAMPLE CLAUSES

While the example clauses described below are described with respect to one particular implementation, it should be understood that, in the context of this document, the content of the example clauses can also be implemented via a method, device, system, and/or another implementation. Additionally, any of examples A-T may be implemented alone or in combination with any other one or more of the examples A-T.

A: A surgical system, including: a scope including: a light source configured to illuminate an operative field in an interior space of a subject's body; and a single camera configured to capture two-dimensional (2D) images of the operative field; a probe configured to move from a first three-dimensional (3D) position in the operative field to a second 3D position in the operative field; an input device configured to receive a first input signal when the probe is disposed at the first 3D position and to receive a second input signal when the probe is disposed at the second 3D position; a display; and at least one processor communicatively coupled to the scope and the input device, the at least one processor being configured to: identify the first 3D position by providing, to a trained machine learning model, at least one first image among the 2D images corresponding to the first input signal; identify the second 3D position by providing, to the trained machine learning model, at least one second image among the 2D images corresponding to the second input signal; determine a distance between the first 3D position and the second 3D position; and cause the display to visually present a third image among the 2D images, a line overlaying the third image and extending between a depiction of the first 3D position and a depiction of the second 3D position in the surgical field, and an indication of the distance.

B: The surgical system of paragraph A, the scope being a first scope, wherein the trained machine learning model was previously trained in a supervised fashion based on: training 2D images of spaces including a surgical instrument, the spaces excluding the operative field, the surgical instrument being different than the probe, the training 2D images being captured by a second scope that is different than the first scope; and sensor data captured by a magnetic field sensor based on positions of a first magnet disposed on a first rod extending from the surgical instrument and positions of a second magnet disposed on a second rod extending from the second scope, the first rod and the second rod being electrically and magnetically insulative.

C: The surgical system of paragraph A, wherein the input device includes a microphone, the first input signal includes a first verbal command from a user holding the scope and/or the probe, and the second input signal includes a second verbal command from the user.

D: A computer-implemented method including: receiving image data captured by a single camera of a first surgical instrument, wherein the image data includes a two-dimensional (2D) image of a second surgical instrument; providing the 2D image to a machine learning model; receiving, from the machine learning model, a three-dimensional (3D) position of the second surgical instrument; determining, based on the 3D position, a feature of the image data; and outputting, to a user, the feature using a surgical assistant user interface.

E: The computer-implemented method of paragraph D, the 2D image being a first 2D image, the 3D position being a first 3D position, the computer-implemented method further including: determining, based on the image data, a second 2D image of the second surgical instrument; providing the second 2D image to the machine learning model; receiving, from the machine learning model, a second 3D position of the second surgical instrument; determining a distance between the first 3D position and the second 3D position; and determining the first feature based on the distance.

F: The computer-implemented method of paragraph D, the 2D image being a first 2D image, the 3D position being a first 3D position, the computer-implemented method further including: determining, based on the image data, a second 2D image and a third 2D image of the second surgical instrument; providing the second 2D image to the machine learning model; receiving, from the machine learning model, a second 3D position of the second surgical instrument; providing the third 3D image to the machine learning model; receiving, from the machine learning model, a third 3D position of the second surgical instrument; determining an angle associated with the first 3D position, the second 3D position, and the third 3D position; and determining the first feature based on the angle.

G: The computer-implemented method of paragraph D, the 2D image being a first 2D image, the 3D position being a first 3D position, the computer-implemented method further including: determining, based on the image data, a second 2D image, a third 2D image, and a fourth 2D image of the second surgical instrument; providing the second 2D image to the machine learning model; receiving, from the machine learning model, a second 3D position of the second surgical instrument; providing the third 2D image to the machine learning model; receiving, from the machine learning model, a third 3D position of the second surgical instrument; providing the fourth 2D image to the machine learning model; receiving, from the machine learning model, a fourth 3D position of the second surgical instrument; determining a region bounded by the first 3D position, the second 3D position, the third 3D position, and the fourth 3D position; and determining the feature based on the region.

H: The computer-implemented method of paragraph D, further including: providing the 3D position to a second machine learning model; receiving, from the second machine learning model, a classification associated with the image data; and determining the feature based on the classification.

I: The computer-implemented method of paragraph H, wherein the classification represents at least one of: a tissue type associated with a tissue depicted by the image data; a physiological structure depicted by the image data; a pathology depicted by the image data.

J: The computer-implemented method of paragraph D, wherein the feature represents a recommended trajectory for moving the second surgical instrument relative to an anatomical part.

K: The computer-implemented method of paragraph D, further including: receiving an anatomical label associated with the 2D image; and determining the feature based on the anatomical label.

L: The computer-implemented method of paragraph K, wherein the feature represents an orientation of the second surgical instrument relative to the anatomical label.

M: The computer-implemented method of paragraph D, wherein the feature represents feedback data about a position of an object relative to an anatomical part, and wherein the position of the object is determined based on the 3D position of the second surgical instrument.

N: A training system, including: a scope configured to capture 2D images of a training space; a first rod extending from the scope; a first sensor configured to detect a first parameter indicative of a 3D position of the first sensor in the training space, the first sensor being mounted on the first rod, the first sensor being disposed away from the scope by a first distance; a tool configured to be disposed in the training space, the 2D images depicting the tool in the training space; a second rod extending from the tool; a second sensor configured to detect a second parameter indicative of a 3D position of the second sensor in the training space, the second sensor being mounted on the second rod, the second sensor being disposed away from the tool by a second distance; at least one processor configured to: determine, based on the first parameter, the 3D position of the first sensor; determine, based on the second parameter, the 3D position of the second sensor; determine, based on the 3D position of the first sensor, a 3D position of the scope; determine, based on the 3D position of the second sensor, a 3D position of the tool; determine, based on the 3D position of the scope and the 3D position of the tool, a ground truth 3D position of the tool relative to the scope; and train a machine learning model by: inputting, into a machine learning model, the 2D images of the training space; receiving, from the machine learning model, a predicted 3D position of the tool relative to the scope; determining a loss between the ground truth 3D position of the tool relative to the scope and the predicted 3D position of the tool relative to the scope; and optimizing parameters of the machine learning model based on the loss.

O: The training system of paragraph N, wherein the scope includes at least one of a laparoscope, an orthoscope, or an endoscope, and wherein the tool includes a surgical instrument.

P: The training system of paragraph N, wherein the first parameter includes a strength of a magnetic field at the 3D position of the first sensor, wherein the second parameter includes a strength of the magnetic field at the 3D position of the second sensor; wherein the scope includes at least one first metal, wherein the tool includes at least one second metal, wherein the first rod includes at least one first insulative material, and wherein the second rod includes at least one second insulative material.

Q: The training system of paragraph P, further including: a magnetic field source configured to emit the magnetic field in the training space, wherein the processor is configured to determine the 3D position of the first sensor based on a position of the magnetic field source and the strength of the magnetic field at the 3D position of the first sensor, and wherein the processor is configured to determine the 3D position of the second sensor based on the position of the magnetic field source and the strength of the magnetic field at the 3D position of the second sensor.

R: The training system of paragraph P, wherein the first insulative material and the second insulative material include at least one of wood or a polymer.

S: The training system of paragraph P, wherein the first distance is in a range of fifteen centimeters (cm) to thirty cm, and wherein the second distance is in a range of fifteen cm to thirty cm.

T: The training system of paragraph P, wherein the machine learning model includes at least one of a convolutional neural network, a residual neural network, a recurrent neural network, or a two-stream fusion network including a color processing stream and a flow stream.

### CONCLUSION

The environments and individual elements described herein may of course include many other logical, programmatic, and physical components, of which those shown in the accompanying figures are merely examples that are related to the discussion herein.

Other architectures may be used to implement the described functionality and are intended to be within the scope of this disclosure. Furthermore, although specific distributions of responsibilities are defined above for purposes of discussion, the various functions and responsibilities might be distributed and divided in different ways, depending on circumstances.

Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

As will be understood by one of ordinary skill in the art, each embodiment disclosed herein can comprise, consist essentially of, or consist of its particular stated element(s), step(s), ingredient(s), and/or component(s). Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiments.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Explicit definitions and explanations used in the present disclosure are meant and intended to be controlling in any future construction unless clearly and unambiguously modified in examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. (canceled)

2. A surgical system, comprising:
a scope comprising:
a light source configured to illuminate an operative field in an interior space of a subject's body; and
a single camera configured to capture two-dimensional (2D) images of the operative field;
a probe configured to move from a first three-dimensional (3D) position in the operative field to a second 3D position in the operative field;
an input device configured to receive a first input signal when the probe is disposed at the first 3D position and to receive a second input signal when the probe is disposed at the second 3D position;
a display; and
at least one processor communicatively coupled to the scope and the input device, the at least one processor being configured to:
identify the first 3D position by providing, to a trained machine learning model, at least one first image among the 2D images corresponding to the first input signal;
identify the second 3D position by providing, to the trained machine learning model, at least one second image among the 2D images corresponding to the second input signal;
determine a distance between the first 3D position and the second 3D position; and
cause the display to visually present a third image among the 2D images, a line overlaying the third image and extending between a depiction of the first 3D position and a depiction of the second 3D position in the surgical field, and an indication of the distance.

3. The surgical system of claim 2, the scope being a first scope, wherein the trained machine learning model was previously trained in a supervised fashion based on:
training 2D images of spaces comprising a surgical instrument, the spaces excluding the operative field, the surgical instrument being different than the probe, the training 2D images being captured by a second scope that is different than the first scope; and
sensor data captured by a magnetic field sensor based on positions of a first magnet disposed on a first rod extending from the surgical instrument and positions of a second magnet disposed on a second rod extending from the second scope, the first rod and the second rod being electrically and magnetically insulative.

4. The surgical system of claim 2, wherein the input device comprises a microphone, the first input signal comprises a first verbal command from a user holding the scope and/or the probe, and the second input signal comprises a second verbal command from the user.

5. A computer-implemented method comprising:
receiving image data captured by a single camera of a first surgical instrument, wherein the image data comprises a two-dimensional (2D) image of a second surgical instrument;
providing the 2D image to a machine learning model;
receiving, from the machine learning model, a three-dimensional (3D) position of the second surgical instrument;
determining, based on the 3D position, a feature of the image data; and
outputting, to a user, the feature using a surgical assistant user interface.

6. The computer-implemented method of claim 5, the 2D image being a first 2D image, the 3D position being a first 3D position, the computer-implemented method further comprising:
determining, based on the image data, a second 2D image of the second surgical instrument;
providing the second 2D image to the machine learning model;
receiving, from the machine learning model, a second 3D position of the second surgical instrument;
determining a distance between the first 3D position and the second 3D position; and
determining the first feature based on the distance.

7. The computer-implemented method of claim 5, the 2D image being a first 2D image, the 3D position being a first 3D position, the computer-implemented method further comprising:
determining, based on the image data, a second 2D image and a third 2D image of the second surgical instrument;
providing the second 2D image to the machine learning model;
receiving, from the machine learning model, a second 3D position of the second surgical instrument;
providing the third 3D image to the machine learning model;
receiving, from the machine learning model, a third 3D position of the second surgical instrument;
determining an angle associated with the first 3D position, the second 3D position, and the third 3D position; and
determining the first feature based on the angle.

8. The computer-implemented method of claim 5, the 2D image being a first 2D image, the 3D position being a first 3D position, the computer-implemented method further comprising:
determining, based on the image data, a second 2D image, a third 2D image, and a fourth 2D image of the second surgical instrument;
providing the second 2D image to the machine learning model;
receiving, from the machine learning model, a second 3D position of the second surgical instrument;
providing the third 2D image to the machine learning model;
receiving, from the machine learning model, a third 3D position of the second surgical instrument;
providing the fourth 2D image to the machine learning model;
receiving, from the machine learning model, a fourth 3D position of the second surgical instrument;
determining a region bounded by the first 3D position, the second 3D position, the third 3D position, and the fourth 3D position; and
determining the feature based on the region.

9. The computer-implemented method of claim 5, further comprising:
providing the 3D position to a second machine learning model;
receiving, from the second machine learning model, a classification associated with the image data; and
determining the feature based on the classification.

10. The computer-implemented method of claim 9, wherein the classification represents at least one of:
a tissue type associated with a tissue depicted by the image data;
a physiological structure depicted by the image data;
a pathology depicted by the image data.

11. The computer-implemented method of claim 5, wherein the feature represents a recommended trajectory for moving the second surgical instrument relative to an anatomical part.

12. The computer-implemented method of claim 5, further comprising:
receiving an anatomical label associated with the 2D image; and
determining the feature based on the anatomical label.

13. The computer-implemented method of claim 12, wherein the feature represents an orientation of the second surgical instrument relative to the anatomical label.

14. The computer-implemented method of claim 5, wherein the feature represents feedback data about a position of an object relative to an anatomical part, and wherein the position of the object is determined based on the 3D position of the second surgical instrument.

15. A training system, comprising:
a scope configured to capture 2D images of a training space;
a first rod extending from the scope;
a first sensor configured to detect a first parameter indicative of a 3D position of the first sensor in the training space, the first sensor being mounted on the first rod, the first sensor being disposed away from the scope by a first distance;
a tool configured to be disposed in the training space, the 2D images depicting the tool in the training space;
a second rod extending from the tool;
a second sensor configured to detect a second parameter indicative of a 3D position of the second sensor in the training space, the second sensor being mounted on the second rod, the second sensor being disposed away from the tool by a second distance;
at least one processor configured to:
determine, based on the first parameter, the 3D position of the first sensor;
determine, based on the second parameter, the 3D position of the second sensor;
determine, based on the 3D position of the first sensor, a 3D position of the scope;
determine, based on the 3D position of the second sensor, a 3D position of the tool;
determine, based on the 3D position of the scope and the 3D position of the tool, a ground truth 3D position of the tool relative to the scope; and
train a machine learning model by:
inputting, into a machine learning model, the 2D images of the training space;
receiving, from the machine learning model, a predicted 3D position of the tool relative to the scope;
determining a loss between the ground truth 3D position of the tool relative to the scope and the predicted 3D position of the tool relative to the scope; and
optimizing parameters of the machine learning model based on the loss.

16. The training system of claim 15, wherein the scope comprises at least one of a laparoscope, an orthoscope, or an endoscope, and
wherein the tool comprises a surgical instrument.

17. The training system of claim 15, wherein the first parameter comprises a strength of a magnetic field at the 3D position of the first sensor,
wherein the second parameter comprises a strength of the magnetic field at the 3D position of the second sensor;
wherein the scope comprises at least one first metal,
wherein the tool comprises at least one second metal,
wherein the first rod comprises at least one first insulative material, and
wherein the second rod comprises at least one second insulative material.

18. The training system of claim 17, further comprising:
a magnetic field source configured to emit the magnetic field in the training space,
wherein the processor is configured to determine the 3D position of the first sensor based on a position of the magnetic field source and the strength of the magnetic field at the 3D position of the first sensor, and
wherein the processor is configured to determine the 3D position of the second sensor based on the position of the magnetic field source and the strength of the magnetic field at the 3D position of the second sensor.

19. The training system of claim 17, wherein the first insulative material and the second insulative material comprise at least one of wood or a polymer.

20. The training system of claim 17, wherein the first distance is in a range of centimeters (cm) to 30 cm, and
wherein the second distance is in a range of 15 cm to 30 cm.

21. The training system of claim 17, wherein the machine learning model comprises at least one of a convolutional neural network, a residual neural network, a recurrent neural network, or a two-stream fusion network comprising a color processing stream and a flow stream.
